(19) United States
(12) Patent Application Publication
(10) Pub. No.: US 2024/0051974 A1
(43) Pub. Date: Feb. 15, 2024

(54) DIRECT REDUCTIVE AMINATIONS WITH MONOTRIFLUOROACETOXY BORANE-AMINES

(71) Applicant: Purdue Research Foundation, West Lafayette, IN (US)

(72) Inventor: Padinjaremadhom V. Ramachandran, West Lafayette, IN (US)

(73) Assignee: Purdue Research Foundation, West Lafayette, IN (US)

(21) Appl. No.: 18/228,829

(22) Filed: Aug. 1, 2023

Related U.S. Application Data
(60) Provisional application No. 63/393,984, filed on Aug. 1, 2022.

(51) Int. Cl. C07F 5/02; C07C 209/28; C07C 213/02; C07D 295/023; C07C 253/30; C07D 207/267; C07D 207/27; C07D 405/06; C07D 211/76; C07D 409/06; C07D 209/46

(52) U.S. Cl. CPC C07F 5/022; C07C 209/28; C07C 213/02; C07D 295/023; C07C 253/30; C07D 207/267; C07D 207/27; C07D 405/06; C07D 211/76; C07D 409/06; C07D 209/46; C07C 2601/14; C07C 2601/08

(57) ABSTRACT

A direct reductive amination of aldehydes, ketones, and keto acids with trifluoroacetoxyborane-amines (TFAB-amines), which are mild reductive amination agents prepared by monoacetoxylation of borane-amines; Aromatic and aliphatic lactams are obtained via a tandem reductive amination-cycloamidation of keto acids using TFAB-amine.

### CROSS-REFERENCE TO RELATED APPLICATION

This application claims priority to U.S. provisional patent application No. 63/393,984, which was filed Aug. 1, 2022, and which is hereby incorporated by reference in its entirety.

### TECHNICAL FIELD

The present disclosure relates to a chemo-selective direct reductive amination of aldehydes, ketones, and keto acids using borane-amine complexes as a mild reductive amination reagent.

### BACKGROUND

Refinement of known reagents to achieve the desired goal is a well-tested, successful strategy in organic synthesis. In area of selective reduction taming of powerful lithium aluminum hydride is achieved by replacing hydrogens with alkoxy groups, whereas the potency of mild metal borohydrides can be increased by replacing hydrides with alkyl groups to obtain super-hydrides. Introduction of electron-withdrawing groups (EWG) to borohydrides affords the milder cyanoborohydride (e.g., NaBH₃CN) or triacetoxyborohydride (e.g., Na(AcO)₃BH), both of which are employed for the selective reduction of imines in the presence of carbonyls. This selectivity is a critical requirement for reductive amination, a fundamentally important, industrial-scale reaction. However, apart from the release of toxic side-products, pH-dependency, use of halogenated solvent, and poor atom economy, the above reagents have a serious limitation when it comes to the reductive amination of aryl carbonyls due to the sluggish generation of imines. Some of these limitations are circumvented by employing amine-boranes, such as pyridine-borane, for reductive amination.

Hence, there is an unmet need for a mild, selective, and direct reductive amination reagent, which overcomes the existing challenges and can carry out reductive amination of challenging carbonyls in one step with excellent yield. It is an object of the present disclosure to provide such a reagent. This and other objects and advantages, as well as inventive features, will be apparent from the detailed description.

### SUMMARY

Provided is a trifluoroacetoxyborane-amine (TFAB-amine) complex as a mild reductive amination reagent for a direct reductive amination of a carbonyl compound in the presence of an amine and a process for their preparation.

A TFAB-amine complex can be prepared from a borane-amine, which is represented by the formula (1):

R₃N—BH₃ (1)

wherein, R₃N is NH₃, NH₂Me, NH₂CH₂Ph, NHMe₂, NH(i-Pr)₂, piperidine, NMe₃, NEt₃, pyridine or 2-picoline.

In exemplary embodiments, a TFAB-amine complex is represented by the formula (2):

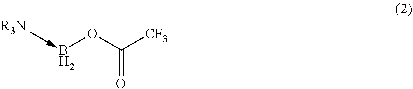

wherein, R₃N is NH₃, NH₂Me, NH₂CH₂Ph, NHMe₂, NH(i-Pr)₂, piperidine, NMe₃, NEt₃, pyridine or 2-picoline.

Provided is a process for preparation of a TFAB-amine complex of the formula (2), which process comprises reacting a borane-amine of formula (1) with a trifluoroacetic acid in the presence of a solvent.

In exemplary embodiments, the most suitable TFAB-amine complex can be TFAB-NH₃ or TFAB-NEt₃.

Examples of solvents that can be used include, but are not limited to, tetrahydrofuran (THF), toluene, and dichloromethane (DCM). The reaction is carried out at a temperature range from about room temperature (RT) to about reflux.

Provided is a process for direct reductive amination of a carbonyl compound, which process comprises reacting the carbonyl compound with an amine in the presence of a TFAB-amine complex.

The carbonyl compounds that can be reductively aminated are aldehydes, ketones, and keto acids. Keto acids can undergo tandem reductive amination-cycloamidation to achieve aromatic and aliphatic lactams. In exemplary embodiments, aldehydes, ketones, and keto acids are represented by formula (3), formula (5), and formula (8), respectively.

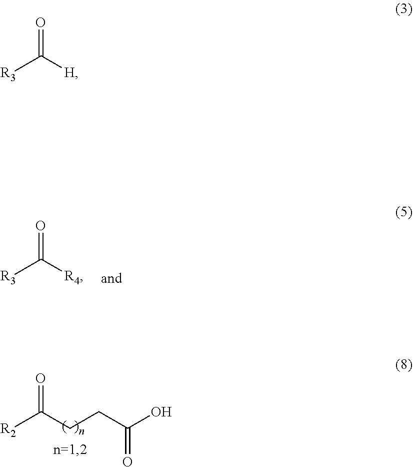

wherein, R₂, R₃, and R₄ are independently selected from the group consisting of hydrogen, alkyl, trifluoromethyl, cycloalkyl, heteroalkyl, heterocycloalkyl, aryl, heteroarylalkyl, arylalkenyl, and arylalkyl, wherein alkyl, cycloalkyl, aryl, and heteroaryl are optionally substituted or R₃ and R₄ are linked via an alkyl chain; and n is 1 or 2.

In exemplary embodiments, amines are represented by the formula (4):

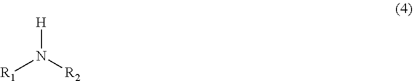

wherein R₁ and R₂ are independently selected from the group consisting of hydrogen, alkyl, trifluoromethyl, cycloalkyl, heteroalkyl, heterocycloalkyl, aryl, heteroaryl, heteroarylalkyl, arylalkenyl, and arylalkyl, wherein alkyl, cycloalkyl, aryl, and heteroaryl are optionally substituted; or R₁ and R₂ are linked via an alkyl chain.

Examples of solvents that can be used for reductive amination include, but are not limited to, THF, toluene, and DCM. The reaction is carried out at a temperature range from about RT to about reflux.

### BRIEF DESCRIPTION OF THE DRAWINGS

The above and other objects, features, and advantages of the present disclosure will be apparent when the description is read in conjunction with the drawings.

FIG. 1 shows aldehydes, ketones, and keto acids used for reductive amination. The aldehydes are numbered as 3a-3e. The ketones are numbered as 5a-5k. The keto acids are numbered as 8a-8i.

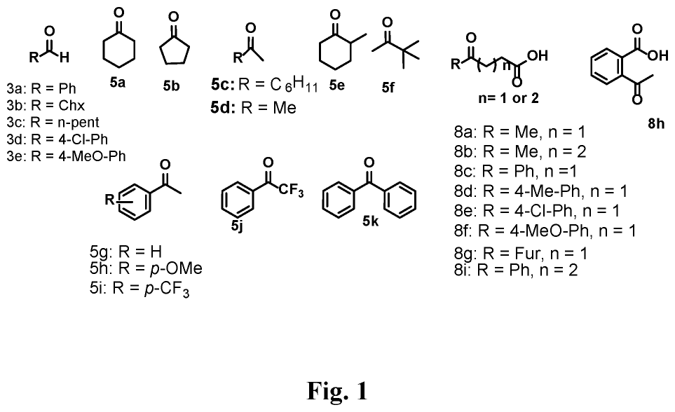

FIG. 2 shows amines used for reductive amination. The amines are numbered 4a-4v. The amino acids are numbered 8a and 8b.

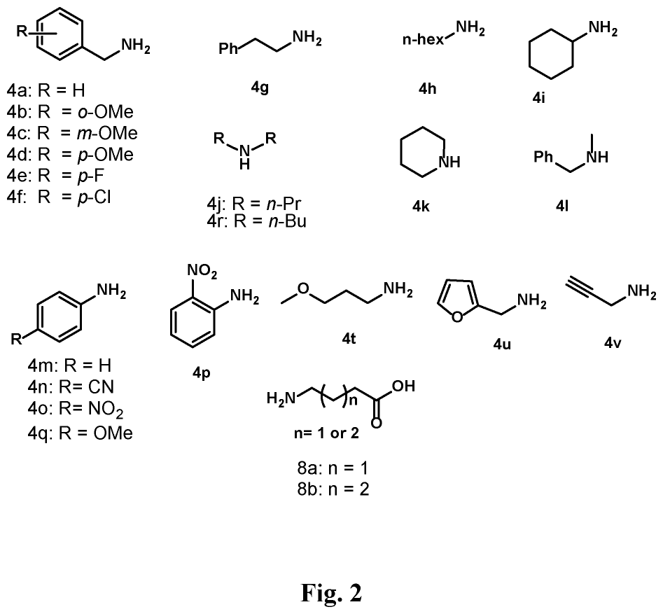

FIG. 3 shows reductive amination of carbonyls using trifluoroacetoxyborane-amine (TFAB-NH₃) in the presence of amines.

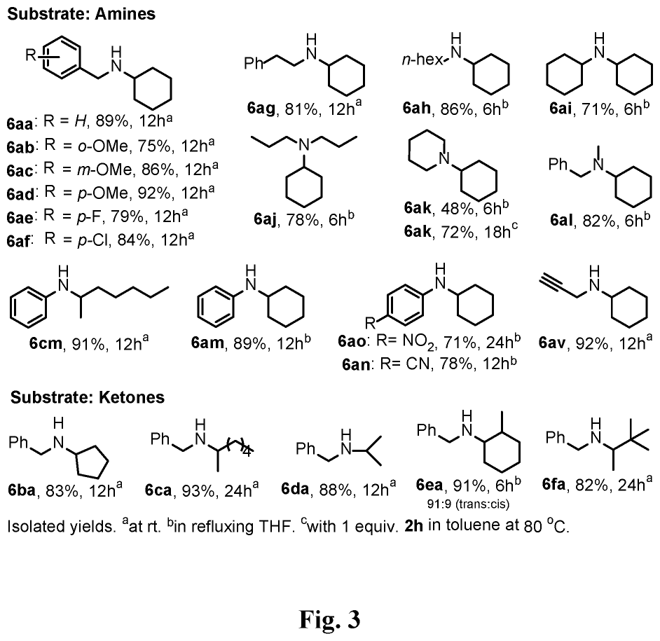

FIG. 4 shows reductive amination of carbonyls using TFAB-NEt₃ in the presence of amines.

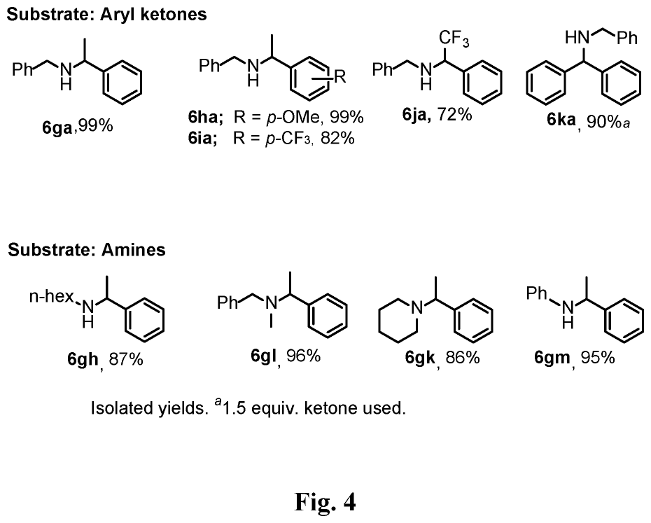

FIG. 5 shows primary amines and secondary amines that can be used in the reductive amination of aldehydes using TFAB-NEt₃ in the presence of amine.

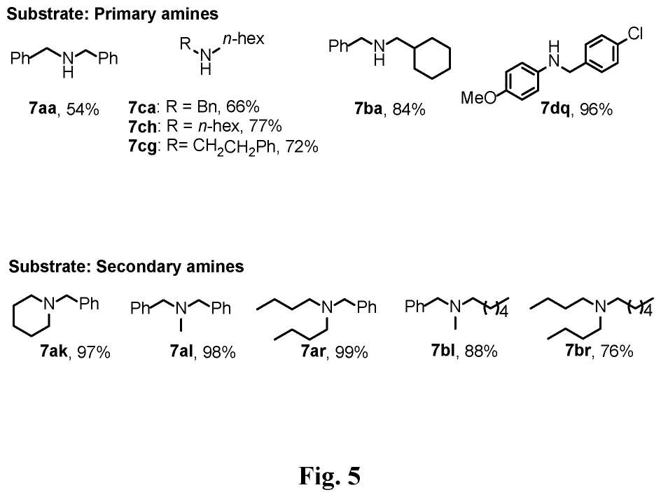

FIG. 6 shows amines that can be used in tandem reductive amination-lactamization of keto acids with TFAB-NH₃.

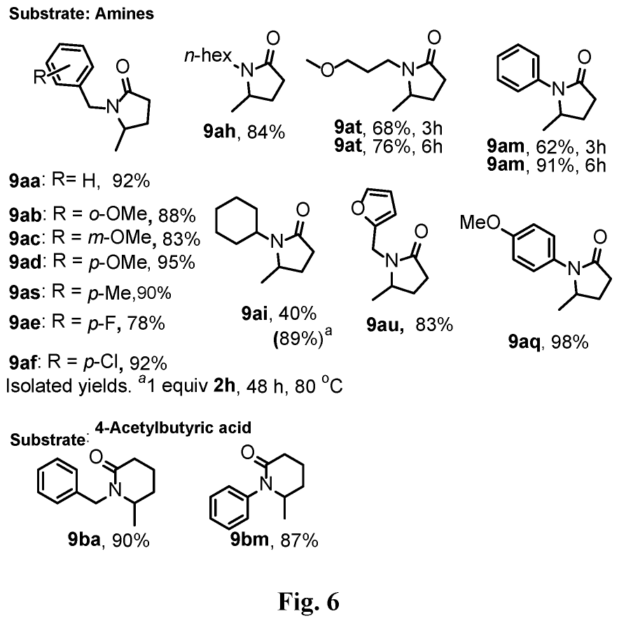

FIG. 7 shows keto acids and amines that can be used in tandem reductive amination-lactamization of keto acids with TFAB-NEt₃.

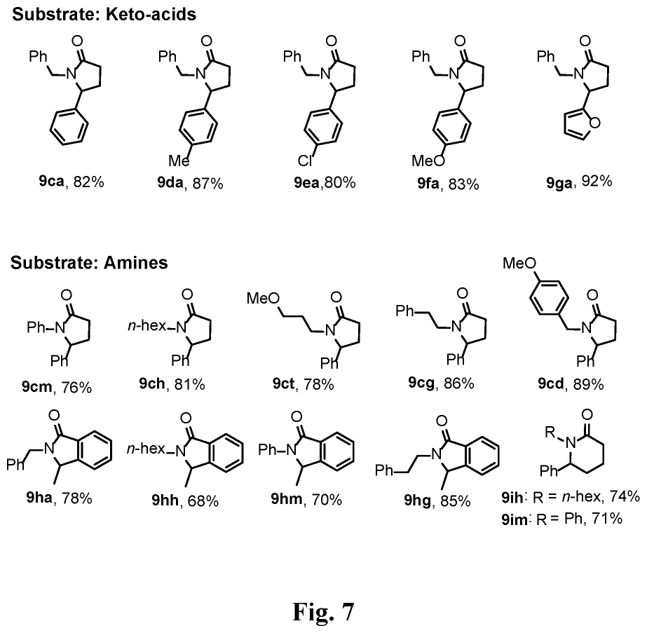

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The “arrow” in a TFAB-amine complex of formula (2) represents the coordinate covalent bond between nitrogen from NR₃ and Boron.

Abbreviations used for the chemical groups are:
NH₃ is ammonia, Me is methyl, CH₂Ph is benzyl, Et is ethyl, i-pr is isopropyl, NMe₃ is trimethyl amine NEt₃ is triethyl amine.

The mildness of the reduction spares functional groups, which are susceptible to hydrogenation or harsh reduction conditions of other reagents. Amine boranes are made milder by attaching trifluoroacetoxy group to boron. Borane-amines undergo exclusive monoacetoxylation to obtain trifluoroacetoxyborane-amines (TFAB-amines) complexes. TFAB-amine complexes are mild, additive-free, and selective direct reductive amination agents with the amine on the reagent influencing chemo selectivity and yields. Compared to well-known reductive amination agents NaBH₃CN and NaBH(OAc)₃, TFAB-amine complexes significantly improve the yields for reductive aminations, including those considered to be challenging. Reductive amination of ketones is always challenging since the ketones will be reduced along with imines. Hence, reductive amination is achieved by either a two-step process in which preparation of imines is achieved before reduction or by hydrogenation of nitriles. TFAB-amine complexes can selectively reduce difficult imines in the presence of carbonyls. TFAB-amine complexes can perform reductive amination of these challenging ketones, such as aryl ketones and keto acids, aldehydes with secondary amines, and deactivated anilines, with improved yield.

In some embodiments, provided is a TFAB-amine complex of formula (2):

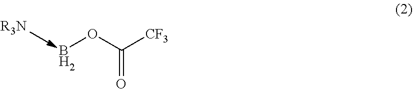

wherein, R₃N is NH₃, NH₂Me, NH₂CH₂Ph, NHMe₂, NH(i-Pr)₂, piperidine, NMe₃, NEt₃, pyridine, or 2-picoline as a mild reductive amination agent for a reductive amination of a carbonyl compound and a process for its preparation.

A TFAB-amine complex of formula (2) can be prepared from an amine borane represented by formula (1):

R₃N—BH₃ (1)

wherein, R₃N is NH₃, NH₂Me, NH₂ CH₂Ph, NHMe₂, NH(i-Pr)₂, piperidine, NMe₃, NEt₃, pyridine or 2-picoline.

In exemplary embodiments, provided is a process for preparation of a TFAB-amine complex of formula (2), which process comprises reacting an amine-borane of formula (1) with a trifluoroacetic acid (TFA) in the presence of a solvent.

TFA reacts with amine-boranes at room temperature (RT) to provide the monoacetoxy derivatives, even with large excess of the acid. Whereas other acids require higher temperature to react and result in the corresponding tris-acyloxy derivatives, TFA provides only the diacetoxy derivatives at higher temperature.

Examples of the solvent that can be used include, but are not limited to, toluene, tetrahydrofuran (THF), and dichloromethane (DCM). The acetoxylation occurs faster in toluene than THF. The reaction can be carried out at different temperatures ranging from about RT to about reflux, such as from RT to about reflux or about RT to reflux.

Examples of amine-boranes can be ammonia-borane (AB) and primary, secondary (sec.), and tertiary (tert.) amine-boranes. The examples of primary amine-boranes include, but are not limited to, ammonia borane (1a), methylamine borane (1b) and benzylamine borane (1c), which were converted to the TFAB-NH₃ (2a), TFAB-methylamine complex (2b) and TFAB-benzylamine complex (2c). The examples of sec. amine-boranes include, but are not limited to, dimethylamine-borane (1d), diisopropylamine-borane (1e) and piperidine-borane (1f), which were converted to TFAB-dimethylamine complex (2d), TFAB-diisopropylamine complex (2e), and TFAB-piperidine complex (2f). The examples of tert. amine-boranes include, but are not limited to, trimethylamine-borane (1g) and triethylamine-borane (1h), which were converted to the TFAB-trimethylamine complex (2g) and TFAB-triethylamine complex (2h). The examples of arylamine-boranes include, but are not limited to, pyridine-borane (1i) and 2-picoline-borane (1j), which were converted to TFAB-pyridine complex (2i) and TFAB-2-picoline complex (2j).

Examples of TFAB-amine complexes are TFAB-NH₃ (2a), TFAB-methylamine complex (2b), TFAB-benzylamine complex (2c), TFAB-dimethylamine complex (2d), TFAB-diisopropylamine complex (2e), TFAB-piperidine complex (2f), TFAB-trimethylamine complex (2g), TFAB-triethylamine complex (2h), TFAB-pyridine complex (2i), and TFAB-2-picoline complex (2j).

The general procedure of synthesis of TFAB-amine complexes of formula (2) is as shown in Scheme 1.

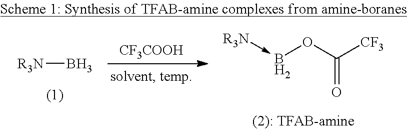

wherein, R₃N is NH₃ (1a), NH₂Me (1b), NH₂CH₂Ph (1C), NHMe₂ (1d), N(i-Pr)₂ (1e), piperidine (1f), NMe₃ (1g), NEt₃ (1h), pyridine (1i) and 2-picoline (1J).

The results of monoacetoxylations are summarized in Table 1 below.

TABLE 1

| R₃N—BH₃ | | Eq. of | Solvent | | Time | | |
|---|---|---|---|---|---|---|---|
| R₃N | | TFA | (1M) | Temp. | (h) | TFAB-amine complex | |
| 1a | Ammonia | 1 eq. | THF | RT | 1 | 2a | CF₃COO—BH₂ |
| | | 1 eq. | DCM | RT | 2 | | —NH₃ |
| | | 1 eq. | Toluene | RT | 1 | | |
| | | 2 eq. | THF | RT | 1 | | |
| | | 3 eq. | THF | RT | 1 | | |
| 1c | Benzylamine | 1 eq. | THF | RT | 18 | 2c | CF₃COO—BH₂—NH₂CH₂Ph |
| | | 1 eq. | Toluene | RT | 2 | | |
| 1b | Methylamine | 1 eq. | THF | RT | 20 | 2b | CF₃COO—BH₂—NH₂Me |
| | | 1 eq. | Toluene | RT | 2 | | |
| 1f | Piperidine | 1 eq. | THF | reflux | 6 | 2f | CF₃COO—BH₂-piperidine |
| | | 1 eq. | Toluene | RT | 4 | | |
| 1d | Dimethylamine | 1 eq. | THE | reflux | 4 | 2d | CF₃COO—BH₂—NHMe₂ |
| | | 1 eq. | Toluene | RT | 4 | | |
| 1e | Diisopropylamine | 1 eq. | THF | reflux | 4 | 2e | CF₃COO—BH₂—NH(i-Pr)₂ |
| | | 1 eq. | Toluene | RT | 4 | | |
| 1g | Trimethylamine | 1.5 eq. | THF | reflux | 12 | 2g | No reaction |
| | | 1.5 eq. | Toluene | RT | 4 | | CF₃COO—BH₂—NEt₃ |
| 1h | Triethylamine | 1.5 eq. | THF | reflux | 12 | 2h | No reaction |
| | | 1.5 eq. | Toluene | RT | 4 | | CF₃COO—BH₂—NMe₃ |
| 1i | Pyridine | 1 eq. | THF | RT | 12 | 2i | CF₃COO—BH₂-Pyridine |
| | | 1 eq. | Toluene | RT | 2 | | |
| 1j | 2-Picoline | 1 eq. | THF | RT | 12 | 2j | CF₃COO—BH₂-2-Picoline |

Provided is a process for direct reductive amination of a carbonyl compound using a TFAB-amine complex, which process comprises reacting the carbonyl compound with an amine in the presence of the TFAB-amine complex.

Examples of the amines that can be used for reductive amination process are amines of formula (4):

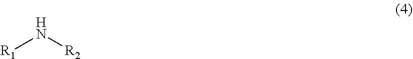

Examples of the carbonyl compounds include, but are not limited to, aldehydes, ketones, and keto acids. In exemplary embodiments, aldehydes can be compounds of formula (3), ketones can be compounds of formula (5), and keto acids can be compounds of formula (8).

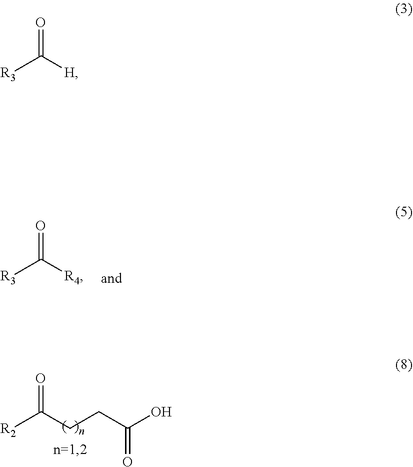

wherein R₁, R₂, R₃ and R₄ are each independently selected from the group consisting of hydrogen, alkyl, trifluoromethyl, cycloalkyl, heteroalkyl, heterocycloalkyl, aryl, heteroaryl, heteroarylalkyl, arylalkenyl, and arylalkyl, wherein alkyl, cycloalkyl, aryl, and heteroaryl are optionally substituted; or R₁ and R₂ are linked via alkyl chain, and n is 1 or 2.

Provided is the product amine of formula (6):

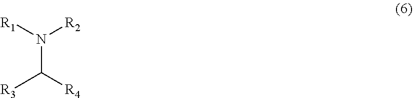

wherein R₁, R₂, R₃ and R₄ are each independently selected from the group consisting of hydrogen, alkyl, trifluoromethyl, cycloalkyl, heteroalkyl, heterocycloalkyl, aryl, heteroaryl, heteroarylalkyl, arylalkenyl, and arylalkyl, wherein alkyl, cycloalkyl, aryl, and heteroaryl are optionally substituted; or R₁ and R₂ are linked via an alkyl chain, obtained by reductive amination of the carbonyl compounds of formula (3), (5), and (8) with the amines of formula (4) in the presence of TFAB-amine complexes of formula (2), wherein formula (2), (3), (4), (5), and (8) are as defined above. Carbonyl compounds and amines are as described herein above.

In exemplary embodiments, the preferred amines can be of formula (4):

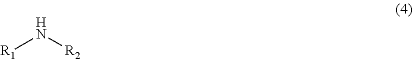

wherein R₁ and R₂ are independently selected from the group consisting of hydrogen, alkyl, cycloalkyl, aryl, heteroarylalkyl, and arylalkyl, wherein alkyl, cycloalkyl, and aryl, are optionally substituted; or R₁ and R₂ are linked via an alkyl chain.

In exemplary embodiments, provided are the preferred aldehydes of formula (3):

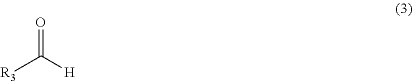

wherein R₃ is hydrogen, alkyl, trifluoroalkyl, cycloalkyl, aryl, or heteroarylalkyl, wherein alkyl, cycloalkyl, and aryl are optionally substituted.

In exemplary embodiments, provided are the preferred ketones of formula (5):

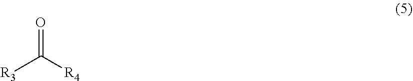

wherein R₃ and R₄ are independently selected from the group consisting of hydrogen, alkyl, trifluoroalkyl, cycloalkyl, aryl, and heteroarylalkyl, wherein alkyl, cycloalkyl, and aryl are optionally substituted.

In exemplary embodiments, provided are the preferred keto acids of formula (8):

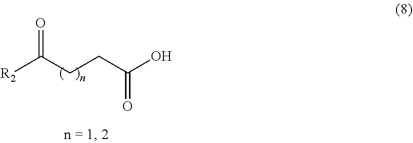

wherein R₂ is hydrogen, alkyl, cycloalkyl, aryl, heteroarylalkyl, or arylalkyl, wherein alkyl, cycloalkyl, and aryl are optionally substituted; and n is 1 or 2.

Alkyl or cycloalkyl is unsubstituted or substituted by one or two of the same or different groups selected from halogen, hydroxy, alkoxy, nitro, nitrile, alkyl, alkenyl, alkynyl, cycloalkyl, cycloalkyl alkyl, aryl, hydroxy alkyl, hydroxy carbonyl, heterocyclylalkyl, heteroarylalkyl, arylalkyl, alkylheteroaryl, cycloalkyl heteroaryl, alkylcarbonyl, alkoxycarbonyl, aryl alkoxycarbonyl, alkylaminocarbonyl, cycloalkylcarbonyl, arylcarbonyl, heteroarylcarbonyl and alkylheteroaryl.

Aryl is unsubstituted or substituted by one or two of the same or different groups selected from halogen, hydroxy, alkoxy, nitro, nitrile, alkyl, alkenyl, alkynyl, cycloalkyl, cycloalkylalkyl, aryl, hydroxy alkyl, hydroxy carbonyl, heteroaryl, heterocyclyl alkyl, heteroarylalkyl, arylalkyl, alkylheteroaryl, cycloalkyl heteroaryl, alkylcarbonyl, alkoxycarbonyl, aryl alkoxycarbonyl, alkylaminocarbonyl, cycloalkylcarbonyl, arylcarbonyl, heteroarylcarbonyl and alkyl heteroaryl.

Heterocyclyl is unsubstituted or substituted by one or two of the same or different groups selected from halogen, hydroxy, alkoxy, nitro, nitrile, alkyl, alkenyl, alkynyl, cycloalkyl, cycloalkyl alkyl, aryl, hydroxy alkyl, hydroxy carbonyl, heteroaryl, heterocyclyl alkyl, heteroaryl alkyl, aryl alkyl, alkyl heteroaryl, cycloalkyl heteroaryl, alkyl carbonyl, alkoxy carbonyl, aryl alkoxycarbonyl, alkylaminocarbonyl, cycloalkylcarbonyl, arylcarbonyl, heteroarylcarbonyl and alkyl heteroaryl.

Examples of solvent that can be used for the reductive amination include, but are not limited to, THF, toluene, and DCM. The suitable solvent used can be THE or toluene.

The reductive amination is carried out at a temperature range of about RT to about reflux, such as about RT to reflux or RT to about reflux.

In some embodiments, provided is the direct reductive amination of aldehydes, ketones or ketoacids, wherein aldehydes, ketones, or ketoacids are as defined herein above, with TFAB-amine complexes such as TFAB-NH₃ (2a) and TFAB-NEt₃ (2h).

The reduction of aldehydes with 2a displayed interesting, unprecedented results. Apart from the expected product alcohol, benzaldehyde (3a) provided a mixture of N,N-dibenzyl- and N,N,N-tribenzylamine, resulting from the reductive alkylation of ammonia in 2a to benzylamine (4a) and further sequential reductive alkylations. Similar results were obtained with cyclohexanecarbaldehyde (3b) as well. Changing the reagent equivalent or solvents to achieve a selective reductive amination of aldehydes, without added external amine, with TFAB-amines 2a, 2c, or 2f as a dual-purpose amine carrier and reduction agent, was not effective. Results are summarized in Table 2 below. Product ratios were determined using ¹HNMR spectroscopy.

TABLE 2

| | | | Product ratios | | | |
|---|---|---|---|---|---|---|
| RCHO | TFAB-amine | | | RCH₂OH | RCH₂NHR′ | RCH₂NR′₂ |
| R | # | amine | R′ | % | % | % |
| Ph | 2a | NH₃ | CH₂Ph | 56 | 34 | 10 |
| Ph | 2c | Benzyl | CH₂Ph | 61 | 21 | 12 |
| | | amine | | | | |
| Ph | 2f | piperidine | piperidine | 62 | — | 37 |
| Cyclohexyl | 2a | NH₃ | cyclohexyl | 75 | 12 | 8 |

Attempted reductive amination of 3a with 4a in the presence of 2a also gave a mixture resulting from the reduction and reductive amination of the carbonyl.

The tandem amine transfer-reductive amination with 2 was absent in ketones. Also, 2a was remarkably slow in reducing ketones, such as cyclohexanone (5a), cyclopentanone (5b), and 2-heptanone (5c) to the corresponding alcohol at RT (<25% reduction in 24 h). Aryl ketones (e.g., acetophenone, 51) were reduced relatively faster (˜₅₀% in 24 h). Other TFAB-amines (2b-2j) were also passive towards ketones, with TFAB-Et₃N (2h) remaining notably unreactive towards aryl ketones as well as various aldehydes. Further investigation revealed that both 2a and 2h efficiently reduced imines to the corresponding amines. For example, N-benzylidenebenzylamine was reduced in quantitative yield by 2a and 2h in THE at RT and in toluene at 60° C., respectively within 6 h. A 1:1 competitive reaction of this imine and 5a with 2a resulted in the exclusive reduction of the imine. Thus, TFAB-amines can be efficient reductive amination agents for the reductive amination of aldehydes and ketones.

The use of TFAB-amine complexes for the reductive amination of aldehydes and ketones were further developed. Scheme 2 illustrates the reductive amination of ketones using 2a.

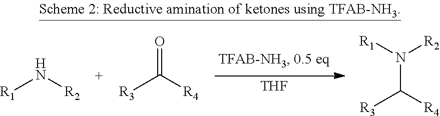

When stirred with benzylamine (4a) and 0.5 equiv. 2a at RT in THE for 12 h, 5a provided 89% yield of N-benzylcyclohexanamine (6aa) without any trace of cyclohexanol (Scheme 2). The atom economy of the reductive amination with complete utilization of the available hydrides is remarkable. Changing the electronic and steric environments of benzylamines with o-, m- and p-substitutions revealed minor changes in product yields from the reductive amination of 5a. Methoxybenzylamines (4b, 4c, 4d) provided the corresponding cyclohexanamines (6ac-6ad) in excellent yields (86%, 75%, and 92% respectively) and electron-poor 4-fluorobenzylamine (4e) and 4-chlorobenzylamine (4f) provided amines 6ae and 6af in 79% and 84% yields, respectively. The scope of this protocol was further examined by treating 5a with aliphatic primary and sec.-amines. Primary amines provided the corresponding N-cyclohexanamines in 71% to 92% yields (6ag-6ai, 6av) and sec.-amines afforded the matching tert.-amines in 48% and 78% yield (6aj-6al). Reaction of ketones 5a and 5c with aniline (4m) produced the corresponding N-alkylated anilines, 6am and 6 cm, in 89% and 91% yields, respectively. Similarly, EWG-bearing 3-aminobenzonitrile (4n) and 4-nitroaniline (4p) provided 78% and 71%, respectively of amines 6an and 6ao, demonstrating the functional group compatibility. The reductive amination of 5a with 2-nitroaniline (4p) using 2a proved challenging, although it was amenable to 2h (vide infra).

The reductive amination of aromatic ketones with TFAB-NEt₃ were carried out as shown in Scheme 3.

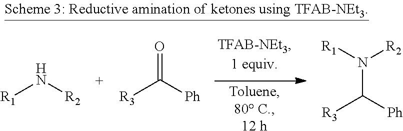

TFAB-NH₃ (2a) was ineffective for the amination of aromatic ketones due to a facile, concurrent reduction to alcohols. However, the milder reagent TFAB-NEt₃ (2h) revealed an excellent propensity for imine reduction. Thus, acetophenone (5g), 4-(trifluoromethyl)acetophenone (5i), 4-methoxyacetophenone (5h), 2,2,2-trifluoroacetophenone (5j) and benzophenone (5k), when stirred with benzylamine (4a) and an equiv. of 2h at 80° C. in toluene, provided the corresponding -amines (6ga-6ka) in 72-99% yields within 12 h. Aniline (4m), and aliphatic primary and sec.-amines (4h, 41, 4k) also gave exceptional yield (86% to 96%) of the corresponding alkylated amines (6gh, 6gl, 6gk, 6 gm) with 5g (Scheme 3). The yield of 6ak was also improved from 40% to 72% using 2h as the reagent. Thus, 2h shows superior selectivity for the reductive amination of carbonyl compounds.

Scheme 4 illustrates the reductive amination of aldehydes with TFAB-NEt₃ as shown in below.

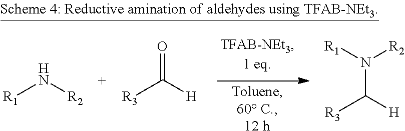

The reductive amination of aldehydes was carried out with 2h knowing that a tert.-amine in the reagent would prevent the alkylation observed with 2a (vide supra). When 3a and 4a were heated at 60° C. with 2h for 12h, dibenzylamine (7aa) was obtained in 54% yield, along with 38% of the tribenzylamine. When reacted with aliphatic aldehydes 3b and 3c, 4a gave the corresponding sec-amines 7ba and 7ca in 84% and 66% yields, respectively, along with traces of the corresponding tert.-amine. Hexanal (3c) and benzylamine (4a) as well as aliphatic amines 4g and 4h yielded the corresponding N-alkylated amines in 72-77% yields. 4-Methoxyaniline (4q) gave the best yields (96% for 7dq) with 4-cholorobenzaldehyde (3d).

The advantage of 2h is more evident from the successful reductive aminations of aldehydes with Sec-amines, such as N-methylbenzylamine (41), N,N-di-n-butylamine (4r), and piperidine (4k), which is challenging due to slow imine formation and faster aldehyde reduction. Benzaldehyde (3a) provided the corresponding Tert.-amines 7al, 7ar, and 7ak in near quantitative (97-99%) yields. Hexanal (3b) also reacted with 41 and 4r to yield the amines 7bl and 7br in 88% and 76% yields, respectively as shown in Scheme 4.

Provided are the results of TFAB-NEt₃ when compared with two of the popular, industrial-scale reductive amination agents, NaBH₃CN and Na(AcO)₃BH. The results are as shown in Scheme 5 below. These commercial reagents afford only 14% and 57% yield, respectively. while a 94% yield of Tert.-amine (7dk) from p-methoxybenzaldehyde and piperidine was achieved with 2h. Reaction of cyclohexanone and 2-nitroaniline with NaBH₃CN failed to provide any N-Benzyl-2-nitroaniline (6ap) after 24h at RT. Although Na(AcO)₃BH provided 30% yield of 6ap, it required the addition of 2-5 equiv of acetic acid (AcOH), two-fold ketone, and 2.8 equiv. of the reagent. However, 2h affords 82% yield within 24 h under standard conditions. Another challenging substrate, acetophenone, provides only 23% and 55%, respectively of the corresponding amines with benzylamine using NaBH₃CN and Na(OAc)₃BH. In contrast, 2h readily affords N-Benzyl-a-methylbenzylamine (6ga) in >99% yield.

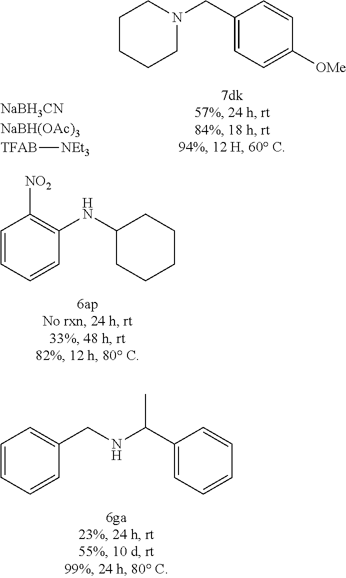

The effective amination of ketones triggered the transition to tandem reductive amination-lactamization of keto acids to prepare functionalized γ- and δ-lactams such as pyrrolidinones and piperidinones. These lactams are well-known vital synthons for natural and unnatural bio-active molecules.

Provided is a process of tandem reductive amination-cyclo-amidation of the keto or amino acids to obtain aromatic and aliphatic lactams of different ring sizes. The process comprises reacting the keto acids with TFAB-amine complexes in presence of amines, wherein the keto acids are represented by formula (8), the TFAB-amine complexes are represented by formula (2) and the product aromatic and aliphatic lactams are represented by formula (9):

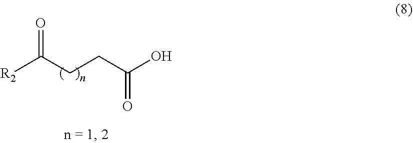

wherein, R₂ is hydrogen, alkyl, trifluoromethyl, cycloalkyl, heteroalkyl, heterocycloalkyl, aryl, heteroaryl, heteroarylalkyl, arylalkenyl, or arylalkyl, wherein alkyl, cycloalkyl, aryl and heteroaryl are optionally substituted; and n is 1 or 2;

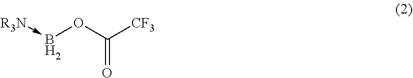

wherein R₃N is NH₃, NH₂Me, NH₂CH₂Ph, NHMe₂, NH(i-Pr)₂, piperidine, NMe₃, NEt₃, pyridine or 2-picoline; and

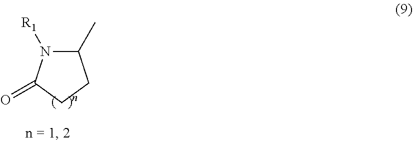

wherein, R₁ is hydrogen, alkyl, trifluoromethyl, cycloalkyl, heteroalkyl, heterocycloalkyl, aryl, heteroaryl, heteroarylalkyl, arylalkenyl, or arylalkyl, wherein alkyl, cycloalkyl, aryl and heteroaryl are optionally substituted; and n is 1 or 2.

The process provides excellent yields of said lactams. Examples of suitable TFAB-amine complexes that can be used include, but are not limited to, TFAB-NH₃ and TFAB-NEt₃. The reactions are illustrated in Schemes 6 and 7 below.

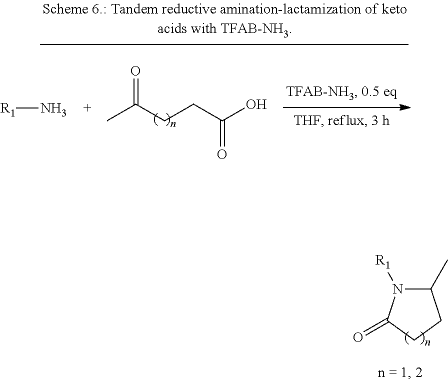

TFAB-NH₃ (2a, 0.5 equiv.) was examined for the reductive amination of keto acid such as levulinic acid (8a) with benzylamine (4a) in refluxing THF, followed by an intra-molecular amidation, when the corresponding N-benzyl-5-methylpyrrolidin-2-one (9aa) was realized in 92% yield within 3 h as shown in Scheme 4. Benzylamines substituted with electron donating groups (4b, 4c, 4d, and 4s) and electron withdrawing groups (4e and 4f) provided 78-92% yield of the respective 5-methylpyrrolidinones. Furfurylamine (5u), n-hexylamine (4h) and 3-methoxypropylamine (4t) gave the lactams 9au, 9ah and 9at in 83%, 84% and 68% yield, respectively. The yield of 9at improved to 76% when the reaction was continued to 6 h. Similarly, aniline (4m) gave a poor yield of pyrrolidinone 9am but improved to 91% in 6 h. 4-Methoxyaniline (4q) provided an excellent (98%) yield of the lactam 9aq. Cyclohexylamine, however, provided only 40% yield of 9ai even after extended reaction. This reaction is amenable to 2h providing an improved yield of 89% in 48 h (Scheme 6). The reductive amination-lactamization process with 2a was extended to 4-acetylbutyric acid (8b) to prepare N-substituted piperidones as well. Thus, 8b reacted with 4a and 4m to give 90% and 87% yield of the isolated N-substituted-6-methylpiperidin-2-ones 9ba and 9bm, respectively.

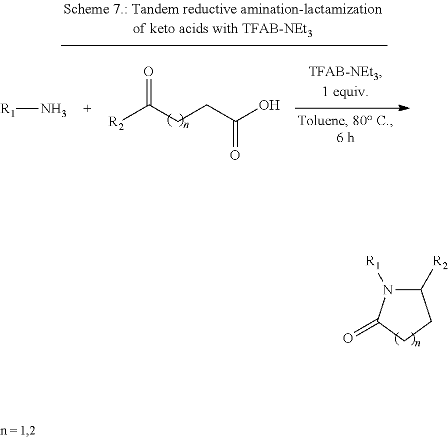

When Keto acid such as benzoylpropanoic acid (8c) was reacted with benzylamine and 2a, a mixture of N-benzyl-5-phenyl-pyrrolidin-2-one and the unsubstituted 5-phenylpyrrolidin-2-one (7:3) was formed. The latter was generated by the earlier mentioned unprecedented reductive alkylation of ammonia in 2a with 8c, followed by lactamization. The reaction was tried with different reaction conditions. The best results were obtained with TFAB-NEt₃ (2h) since the tert-amine cannot participate in reductive amination. Substituted benzoylpropanoic acids (8c-8f) and 4-(2-furyl)-4-oxobutanoic acid (8g) were aminated with 4a, followed by cyclization to form pyrrolidones (9ca-9ga) in 80-92% yields with 2h. Substituted benzylamine (4d), aliphatic amines (4g), (4h), and (4t), and aniline (4m) also gave similar yields of corresponding lactams with 8c. 2-Acetylbenzoic acid (8h) reacted with benzylamine (4a), n-hexylamine (4h), aniline (4m) and 2-phenylethylamine (4g) to provide 9ha, 9hh, 9hm, and 9hg, respectively in 78-92% yields. Furthermore, benzoyl butanoic acid (8i) reacted with 4m and 4h to give substituted 2-piperidones 9im and 9ih in 71% and 74% yields, respectively. The products were readily isolated, without any chromatographic purification.

2a and 2h are selective and effective reductive amine agents for all aldehydes, ketones or ketoacids including challenging carbonyls, such as aldehydes with secondary amines as well as aryl ketones, and deactivated anilines.

Provided is a process for chiral direct reductive amination of a carbonyl compound for preparation of a chiral amine. The process comprises reacting the carbonyl compound with an amine in the presence of an asymmetric TFAB-amine complex, wherein the asymmetric TFAB-amine complex represented by formula (2′):

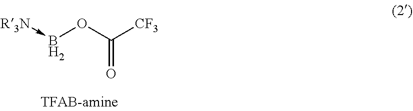

wherein R′₃ is selected from the group consisting of alkyl, cycloalkyl, heteroalkyl, heterocycloalkyl, heteroarylalkyl, arylalkenyl, and arylalkyl, wherein the said group comprises chiral centers, and alkyl, cycloalkyl, aryl and heteroaryl are optionally substituted.

Alkyl or cycloalkyl is unsubstituted or substituted by one or two of the same or different groups selected from halogen, hydroxy, alkoxy, nitro, nitrile, alkyl, alkenyl, alkynyl, cycloalkyl, cycloalkyl alkyl, aryl, hydroxy alkyl, hydroxy carbonyl, heterocyclylalkyl, heteroarylalkyl, arylalkyl, alkylheteroaryl, cycloalkyl heteroaryl, alkylcarbonyl, alkoxycarbonyl, aryl alkoxycarbonyl, alkylaminocarbonyl, cycloalkylcarbonyl, arylcarbonyl, heteroarylcarbonyl and alkylheteroaryl.

Aryl is unsubstituted or substituted by one or two of the same or different groups selected from halogen, hydroxy, alkoxy, nitro, nitrile, alkyl, alkenyl, alkynyl, cycloalkyl, cycloalkylalkyl, aryl, hydroxy alkyl, hydroxy carbonyl, heteroaryl, heterocyclyl alkyl, heteroarylalkyl, arylalkyl, alkylheteroaryl, cycloalkyl heteroaryl, alkylcarbonyl, alkoxycarbonyl, aryl alkoxycarbonyl, alkylaminocarbonyl, cycloalkylcarbonyl, arylcarbonyl, heteroarylcarbonyl and alkyl heteroaryl.

Heterocyclyl is unsubstituted or substituted by one or two of the same or different groups selected from halogen, hydroxy, alkoxy, nitro, nitrile, alkyl, alkenyl, alkynyl, cycloalkyl, cycloalkyl alkyl, aryl, hydroxy alkyl, hydroxy carbonyl, heteroaryl, heterocyclyl alkyl, heteroaryl alkyl, aryl alkyl, alkyl heteroaryl, cycloalkyl heteroaryl, alkyl carbonyl, alkoxy carbonyl, aryl alkoxycarbonyl, alkylaminocarbonyl, cycloalkylcarbonyl, arylcarbonyl, heteroarylcarbonyl and alkyl heteroaryl.

Thus, TFAB-amine complexes are mild, additive-free, direct reductive amination agents for aldehydes, ketones, and keto acids. Compared to popular reductive amination agents such as NaBH₃CN and NaBH(OAc)₃, TFAB-amine complexes particularly TFAB-NEt₃ significantly improves the yields for reductive aminations, including those considered challenging carbonyls, such as aldehydes with secondary amines as well as aryl ketones, and deactivated anilines. Keto acids are readily transformed to lactams via a tandem reductive amination-cyclo-amidation.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

The term “substituted”(e.g., as in “optionally substituted”) refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, and carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” refers to substituted or unsubstituted straight chain and branched alkyl groups and cycloalkyl groups having from 1 to about 20 carbon atoms (C₁-C₂0), 1 to 12 carbons (C₁-C₁₂), 1 to 8 carbon atoms (C₁-C₅), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” refers to substituted or unsubstituted straight chain and branched divalent alkenyl and cycloalkenyl groups having from 2 to 20 carbon atoms(C₂-C₂0), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

An alkynyl group is a substituent, which contains an open point of attachment on a carbon atom that would form if a hydrogen atom bonded to a triply bonded carbon is removed from the molecule of an alkyne. The term “hydroxyalkyl” refers to alkyl groups as defined herein and substituted with at least one hydroxyl (—OH) group.

The term “cycloalkyl” refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and cryloyl groups and the like. When the group containing the carbon atom that is bonded to the carbonyl carbon atom contains a halogen, the group is termed a “haloacyl” group. An example is a trifluoroacetyl group.

The term “aryl” refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” refers to substituted or unsubstituted aromatic and non-aromatic ring compounds containing three or more ring members, of which one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups can include 3 to 8 carbon atoms (C₃-C₅), 3 to 6 carbon atoms (C₃-C₆) or 6 to 8 carbon atoms (C₆-C₅).

A heteroaryl ring is an embodiment of a heterocyclyl group. The phrase “heterocyclyl group” includes fused ring species including those that include fused aromatic and non-aromatic groups. Representative heterocyclyl groups include, but are not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” refers to primary, secondary, and tertiary amines having, e.g., the formula N(group)3 wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” refers to a substituent of the form —NH₂, —NHR, —NR₂, —NR₃+, wherein each R is independently selected, and protonated forms of each, except for —NR₃+, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” and “halide” group, by themselves or as part of another substituent, mean, unless otherwise stated, a fluorine, chlorine, bromine, or iodine atom. The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)2 and the like.

The terms “optionally substituted” and “optional substituents” are used to describe groups, which are either unsubstituted or substituted with one or more of the substituents specified. When the groups in question are substituted with more than one substituent, the substituents can be the same or different. The terms “independently” “independently are” and “independently selected from” mean that the groups in question may be the same or different. Certain of the defined groups or substituents can occur more than once in the structure, and upon such occurrence each group or substituent shall be defined independently of the other.

With various embodiments have been described, it will be apparent to those of ordinary skill in the art that many more embodiments and implementations are possible. Accordingly, the embodiments described herein are examples, not the only possible embodiments and implementations.

### Experimental

General Information: ¹¹B, ¹⁹F, ¹³C, and ¹H NMR spectra were recorded at room temperature, on a Varian INOVA 300 MHz NMR spectrophotometer. Chemical shifts (6 values) are reported in parts per million relative to BF₃.Et₂O {Peterson, 2002 #173} or ¹¹B NMR spectra. PMR spectral data are reported as: δ value, multiplicity (s=singlet, d=doublet, t=triplet, q=quartet, p=pentet, h=hextet, m=multiplet, br=broad) with integration. All solvents for routine isolation of products were reagent-grade. All amines and carbonyls were purchased from commercial sources and used without further purification.

Synthesis of TFAB-ammonia complex:

Anhydrous THE (3 mL) was added to ammonia borane (3 mmol, 1 equiv.) in a 15 mL round bottom flask. Trifluoroacetic acid (3 mmol, 1 equiv.) was added to the above solution at 0° C. and the resulting reaction mixture was stirred for 1 hour at room temperature. The completion of reaction was confirmed by ¹¹B-NMR spectroscopy as indicated by the disappearance of the 6-22 ppm peak due to ammonia borane and the appearance of δ −5 ppm peak due to TFAB-NH₃.

Preparation of Crystal for X-ray crystallography:

1.5 mmol of 18-crown-6 was added to 1M THF solution containing 3 mmol of TFAB-NH₃. The mixture was stirred for 5 min and filtered. The clear solution was kept in a small vial inside a larger vial containing hexane at RT for a week to get colorless transparent block crystals. Details of crystal structure are described in X-Ray crystallography data (vide infra).

Synthesis of Trifluoroacetoxyboarae-amine (TFAB-amine complex):

Anhydrous toluene (3 mL) was added to the respective amine boranes (3 mmol, 1 equiv.) in a 15 mL round bottom flask. Trifluoroacetic acid (4.5 mmol, 1.5 equiv.) was added to the above solution at 0° C. and the resulting reaction mixture was stirred at room temperature for 4-6 h. The completion of reaction was confirmed by 11B-NMR spectroscopy as indicated by the disappearance of the amine borane peak (δ−13 to −19 ppm) and appearance of the TFAB-amine peak (S 0 to −5 ppm). Excess trifluoroacetic acid was then removed in vacuo to obtain the TFAB-amine complex.

Reductive amination of ketones with TFAB-ammonia:

Procedure A: To a 1M solution of TFAB-ammonia (3.3 mmol, 1.1 equiv.) in THF, ketone (6 mmol, 2 equiv.) and the amine (6 mmol, 2 equiv.) were added. The reaction mixture was stirred for 12-24 hrs at room temperature. Upon completion of the reaction, as revealed by TLC, the resulting mixture was quenched with NaOH (5 mL, 3 M), diluted with water (10 mL), and extracted with diethyl ether (3×15 mL). The combined organic extracts were washed with brine (1×45 mL), dried over anhydrous Na₂SO₄ and concentrated in vacuo to obtain the crude product, which was purified by silica gel flash chromatography to obtain the desired product.
Procedure B: To a 1M solution of TFAB-ammonia (3.3 mmol, 1.1 equiv.) in THF, ketone (9 mmol, 3 equiv.) and the amine (6 mmol, 2 equiv.) were added. The reaction mixture was refluxed for 6-12 hrs. Upon completion of the reaction, as revealed by TLC, the resulting mixture was cooled to RT and quenched with NaOH (5 mL, 3 M), diluted with water (10 mL), and extracted with diethyl ether (3×15 mL). The combined organic extracts were washed with brine (1×45 mL), dried over anhydrous Na₂SO₄ and concentrated in vacuo to obtain the crude product, which was purified by silica gel flash chromatography to obtain the desired product.

Reductive amination of aldehydes with TFAB-NEt₃

Procedure C: To a 1M solution of TFAB-NEt₃ (3.3 mmol, 1.1 equiv.) in Toluene, aldehyde (3 mmol, 1 equiv.) and the amine (3.3 mmol, 1.1 equiv.) were added. The reaction mixture was stirred at 60° C. for 12 h. Upon completion of the reaction, as revealed by TLC, the resulting mixture was cooled to RT and quenched with NaOH (5 mL, 3 M), diluted with water (10 mL), and extracted with diethyl ether (3×15 mL). The combined organic extracts were washed with brine (1×45 mL), dried over anhydrous Na₂SO₄ and concentrated in vacuo to obtain the crude product, which was purified by silica gel flash chromatography to obtain the desired product.

Reductive amination of aromatic ketones with TFAB-NEt₃

Procedure D: To a 1M solution of TFAB-NEt₃ (3.3 mmol, 1.1 equiv.) in Toluene, ketone (3 mmol, 1 equiv.) and the amine (3 mmol, 1 equiv.) were added. The reaction mixture was stirred at 80° C. for 12-18 h. Upon completion of the reaction, as revealed by TLC, the resulting mixture was cooled to RT and quenched with NaOH (5 mL, 3 M), diluted with water (10 mL), and extracted with diethyl ether (3×15 mL). The combined organic extracts were washed with brine (1×45 mL), dried over anhydrous Na₂SO₄ and concentrated in vacuo to obtain the crude product, which was purified by silica gel flash chromatography to obtain the desired product.

Tandem reductive amination-lactamization of levulinic acid with TFAB-ammonia

Procedure E: To a 1M solution of TFAB-ammonia (3.3 mmol, 1.1 equiv) in THF, the keto-acid (6 mmol, 2 equiv.) and the amine (9 mmol, 3 equiv.) were added. The reaction mixture was refluxed for 3-6 hrs. Upon completion of the reaction, as revealed by TLC, the resulting mixture was cooled to RT and quenched with a saturated NaHCO₃ solution (5 mL), diluted with water (10 mL), and extracted with diethyl ether (3×15 mL). The combined organic extracts were washed with brine (1×45 mL), dried over anhydrous Na₂SO₄ and concentrated in vacuo to obtain the crude product, which was purified by silica gel flash chromatography to obtain the desired product.

Tandem-reductive amination-lactamization of keto-acid by TFAB-NEts

Procedure F: To a 1M solution of TFAB-NEt₃ (3.3 mmol, 1.1 equiv) in toluene, the keto-acid (3 mmol, 1 equiv.) and the amine (4.5 mmol, 1.5 equiv.) were added. The reaction mixture was stirred at 80° C. for 6 hrs. Upon completion of the reaction, as revealed by TLC, the resulting mixture was cooled to RT and quenched with a saturated NaHCO₃ solution (5 mL), diluted with water (10 mL), and extracted with diethyl ether (3×15 mL). The combined organic extracts were washed twice with 2M HCl. The organic layer was dried over anhydrous Na₂SO₄ and concentrated in vacuo to obtain the crude product.

Tandem-reductive amination-lactamization of amino-acids and aldehydes by TFAB-NEt₃

Procedure G: To a 1M solution of TFAB-NEt₃ (3.3 mmol, 1.1 equiv) in toluene, the amino-acid (4.5 mmol, 1.5 equiv.) and the aldehyde (3 mmol, 1 equiv.) were added. The reaction mixture was stirred at 60° C. for 16-48 hrs. Upon completion of the reaction, as revealed by TLC, the resulting mixture was cooled to RT and quenched with a saturated NaHCO₃ solution (5 mL), diluted with water (10 mL), and extracted with diethyl ether (3×15 mL). The combined organic extracts were washed twice with 2M HCl. The organic layer was dried over anhydrous Na₂SO₄ and concentrated in vacuo to obtain the crude product which was purified by silica gel flash chromatography to obtain the desired product.

Tandem-reductive amination-lactamization of amino-acids and ketones by TFAB-NEt₃

Procedure H: To a 1M solution of TFAB-NEt₃ (3.3 mmol, 1.1 equiv) in toluene, the amino-acid (3 mmol, 1 equiv.) and the ketone (4.5 mmol, 1.5 equiv.) were added. The reaction mixture was stirred at 80° C. for 16-48 hrs. Upon completion of the reaction, as revealed by TLC, the resulting mixture was cooled to RT and quenched with a saturated NaHCO₃ solution (5 mL), diluted with water (10 mL), and extracted with diethyl ether (3×15 mL). The combined organic extracts were washed twice with 2M HCl. The organic layer was dried over anhydrous Na₂SO₄ and concentrated in vacuo to obtain the crude product which was purified by silica gel flash chromatography to obtain the desired product.

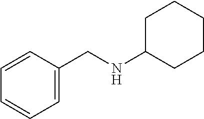 [SMILES: c1ccc(CNC2CCCCC2)cc1]

### N-benzylcyclohexanamine (6aa)

The product was prepared using procedure A and isolated in 89% yield (1.0 g, 5.34 mmol) as a pale yellow liquid after purification via column chromatography on silica gel using hexane/EtOAc 95:5 as an eluent.

¹H NMR (300 MHz, CDCl₃) δ 7.37-7.05 (m, 5H), 3.77 (d, J=1.2 Hz, 2H), 2.45 (tt, J=10.0, 3.6 Hz, 1H), 1.96-1.83 (m, 2H), 1.80-1.55 (m, 3H), 1.35-1.00 (m, 5H).

¹³C NMR (75 MHz, CDCl₃) δ 139.4, 132.3, 129.3, 128.4, 56.2, 50.3, 33.7, 26.3, 25.1.

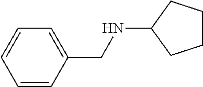 [SMILES: c1ccc(CNC2CCCC2)cc1]

### N-benzylcyclopentanamine (6ba)

The product was prepared using procedure A and isolated in 83% yield (0.871 g, 4.98 mmol) as a colourless liquid after purification via column chromatography on silica gel using hexane/EtOAc 95:5 as an eluent.

¹H NMR (300 MHz, Chloroform-d) δ 7.25 (d, J=8.9 Hz, 5H), 3.72 (d, J=9.4 Hz, 2H), 3.06 (p, J=6.6 Hz, 1H), 1.95-1.20 (m, 8H).

¹³C NMR (75 MHz, CDCl₃) δ 138.9, 132.4, 129.4, 128.4, 59.2, 52.0, 33.2, 24.2.

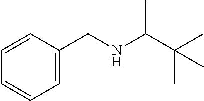

### N-benzyl-3,3-dimethylbutan-2-amine (6fa)

The product was prepared using procedure A and isolated in 82% yield (0.935 g, 4.92 mmol) as a colourless liquid after purification via column chromatography on silica gel using hexane/EtOAc 95:5 as an eluent.

¹H NMR (300 MHz, CDCl₃) δ 7.40-7.18 (m, 5H), 3.94 (d, J=13.2 Hz, 1H), 3.66 (d, J=13.2 Hz, 1H), 2.36-2.24 (m, 1H), 1.09 (d, J=6.4 Hz, 3H), 0.87 (s, 9H).

¹³C NMR (75 MHz, CDCl₃) δ 141.2, 128.2, 128.1, 126.6, 61.2, 52.7, 34.5, 26.6, 14.8.

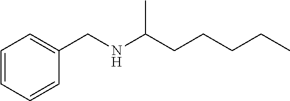

### N-benzyl-3,3-dimethylbutan-2-amine (6ca)

The product was prepared using procedure A and isolated in 93% yield (1.148 g, 5.58 mmol) as a pale yellow liquid after purification via column chromatography on silica gel using hexane/EtOAc 95:5 as an eluent.

¹H NMR (300 MHz, CDCl₃) δ 7.32 (d, J=4.3 Hz, 4H), 7.28-7.17 (m, 1H), 4.18-3.40 (m, 2H), 2.89-2.29 (m, 1H), 1.56-1.38 (m, 1H), 1.42-1.18 (m, 7H), 1.08 (d, J=6.2 Hz, 3H), 0.94-0.81 (t, J=7.0 Hz, 3H).

¹³C NMR (75 MHz, CDCl₃) δ 140.8, 128.3, 128.0, 126.7, 52.6, 51.5, 37.2, 32.2, 25.8, 22.8, 20.5, 14.2.

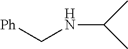

### N-benzylpropan-2-amine (6da)

The product was prepared using procedure A and isolated in 88% yield (0.787 g, 5.3 mmol) as a yellow liquid after purification via column chromatography on silica gel using hexane as an eluent.

¹H NMR (300 MHz, Chloroform-d) δ 7.38-7.20 (m, 5H), 3.79 (s, 2H), 2.86 (p, J=6.2 Hz, 1H), 1.69 (s, 1H), 1.11 (d, J=6.2 Hz, 6H).

¹³C{1H} NMR (75 MHz, CDCl₃) δ 134.9, 129.3, 128.6, 128.0, 49.3, 48.1, 20.6.

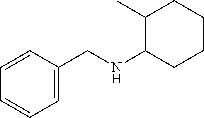

### N-benzyl-2-methylcyclohexan-1-amine (6ea)

The product was prepared using procedure A and isolated in 91% yield as a trans/cis (91:9) mixture (1.108 g, 5.5 mmol) as a yellow liquid after purification via column chromatography on silica gel using hexane as an eluent.

¹H NMR (300 MHz, CDCl₃) δ 7.65-6.98 (m, 5H), 3.96-3.64 (m, 2H), 2.65 (dt, J=8.0, 4.0 Hz, 1H), 1.96 (dq, J=6.7, 3.4 Hz, 1H), 1.81-1.17 (m, 8H), 0.95 (t, J=6.8 Hz, 3H).

¹³C NMR (75 MHz, CDCl₃) δ 141.1, 128.2, 128.0, 126.6, 57.7, 51.2 33.0, 31.0, 28.4, 23.6, 22.5, 14.1.

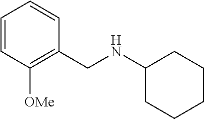 [SMILES: COc1ccccc1CNC1CCCCC1]

### N-(2-methoxybenzyl)cyclohexanamine (6ab)

The product was prepared using procedure A and isolated in 75% yield (0.977 g, 4.5 mmol) as a orange-yellow liquid after purification via column chromatography on silica gel using hexane/EtOAc 95:5 as an eluent.

¹H NMR (300 MHz, CDCl₃) δ 7.29-7.16 (m, 2H), 6.96-6.81 (m, 2H), 3.82 (d, J=6.6 Hz, 5H), 2.43 (tt, J=9.7, 3.6 Hz, 1H), 1.98-1.84 (m, 2H), 1.80-1.54 (m, 4H), 1.35-1.03 (m, 5H).

¹³C NMR (75 MHz, CDCl₃) δ 157.5, 129.6, 128.8, 127.9, 120.3, 110.1, 55.9, 55.3, 46.3, 33.6, 26.3, 25.1.

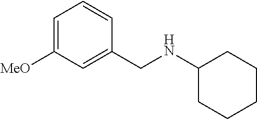 [SMILES: COc1cccc(CNC2CCCCC2)c1]

### N-(3-methoxybenzyl)cyclohexanamine (6ac)

The product was prepared using procedure A and isolated in 86% yield (1.130 g, 5.2 mmol) as a yellow liquid after purification via column chromatography on silica gel using hexane/EtOAc 95:5 as an eluent.

¹H NMR (300 MHz, CDCl₃) δ 7.26-7.67 (m, 4H), 3.8 (m, 5H), 2.4-2.5 (m, 1H), 1.96-1.83 (m, 2H), 1.80-1.55 (m, 3H), 1.35-1.00 (m, 5H).

¹³C NMR (75 MHz, CDCl₃) δ 159.6, 142.6, 129.3, 120.3, 56.2, 55.2, 51.1, 33.7, 26.3, 25.1.

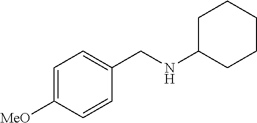 [SMILES: COc1ccc(CNC2CCCCC2)cc1]

### N-(4-methoxybenzyl)cyclohexanamine (6ad)

The product was prepared using procedure A and isolated in 92% yield (1.314 g, 5.5 mmol) as a colourless liquid after purification via column chromatography on silica gel using hexane/EtOAc 95:5 as an eluent.

¹H NMR (300 MHz, CDCl₃) δ 7.30-7.18 (m, 2H), 6.94-6.77 (m, 2H), 3.79 (s, 3H), 3.74 (s, 2H), 2.47 (tt, J=10.0, 3.7 Hz, 1H), 1.98-1.84 (m, 2H), 1.80-1.67 (m, 2H), 1.68-1.54 (m, 1H), 1.33-0.92 (m, 5H).

¹³C NMR (75 MHz, CDCl₃) δ 158.3, 133.0, 129.2, 113.7, 56.1, 55.3, 50.5, 33.6, 26.3, 25.1.

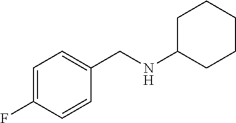 [SMILES: Fc1ccc(CNC2CCCCC2)cc1]

### N-(4-fluoro)cyclohexanamine (6ae)

The product was prepared using procedure A and isolated in 79% yield (0.973 g, 4.7 mmol) as a colorless liquid after purification via column chromatography on silica gel using hexane/EtOAc 95:5 as an eluent.

¹H NMR (300 MHz, CDCl₃) δ 7.34-7.20 (m, 2H), 7.08-6.91 (m, 2H), 3.76 (s, 2H), 2.60-2.38 (m, 1H), 1.96-1.53 (m, 5H), 1.28-1.11 (m, 5H).

¹³C NMR (75 MHz, CDCl₃) δ 163.3, 160.0, 136.6, 129.5, 129.4, 115.2, 114.9, 56.2, 50.4, 33.7, 26.3, 25.1.

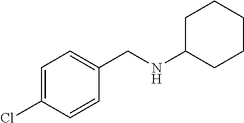 [SMILES: Clc1ccc(CNC2CCCCC2)cc1]

### N-(4-chloro)cyclohexanamine (6af)

The product was prepared using procedure A and isolated in 84% yield (1.124 g, 5 mmol) as a colorless liquid after purification via column chromatography on silica gel using hexane/EtOAc 95:5 as an eluent.

¹H NMR (300 MHz, Chloroform-d) δ 7.52-7.09 (m, 4H), 3.77 (s, 2H), 2.45 (tt, J=10.1, 3.7 Hz, 1H), 1.89 (dd, J=11.8, 3.8 Hz, 2H), 1.81-1.69 (m, 2H), 1.66-1.54 (m, 1H), 1.38-0.87 (m, 5H).

¹³C{1H} NMR (75 MHz, CDCl₃) δ 139.4, 132.3, 129.3, 128.3, 56.1, 50.3, 33.6, 26.2, 25.1.

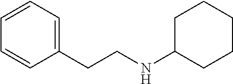 [SMILES: c1ccc(CCNC2CCCCC2)cc1]

### N-phenethylcyclohexanamine (6ag)

The product was prepared using procedure A and isolated in 81% yield (0.987 g, 4.9 mmol) as a colorless liquid after purification via column chromatography on silica gel using hexane/EtOAc 95:5 as an eluent.

¹H NMR (300 MHz, CDCl₃) δ 7.43-7.10 (m, 5H), 3.03-2.70 (m, 4H), 2.42 (tt, J=10.3, 3.7 Hz, 1H), 2.01-1.50 (m, 5H), 1.47-0.90 (m, 5H).

¹³C NMR (75 MHz, CDCl₃) δ 140.1, 128.6, 128.4, 126.0, 56.8, 48.4, 36.8, 33.7, 26.3, 25.2.

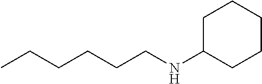

### N-hexylcyclohexanamine (6ah)

The product was prepared using procedure B and isolated in 86% yield (0.952 g, 5.2 mmol) as a colorless liquid after purification via column chromatography on silica gel using hexane/EtOAc 95:5) as an eluent.

¹H NMR (300 MHz, CDCl₃) δ 2.57 (t, J=7.2 Hz, 2H), 2.36 (tt, J=10.3, 3.7 Hz, 1H), 1.92-1.52 (m, 2H), 1.42 (q, J=7.5 Hz, 2H), 1.36-1.20 (m, 3H), 1.25-0.90 (m, 12H), 0.90-0.77 (m, 3H).

¹³C NMR (75 MHz, CDCl₃) δ 57.0, 47.2, 33.8, 31.9, 30.6, 27.3, 26.3, 25.2, 22.7, 14.2.

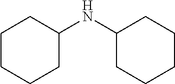 [SMILES: C1CCC(NC2CCCCC2)CC1]

### N-hexylcyclohexanamine (6ai)

The product was prepared using procedure B and isolated in 71% yield (0.771 g, 4.26 mmol) as a colorless liquid after purification via column chromatography on silica gel using hexane the eluent.

¹H NMR (300 MHz, CDCl₃) δ 2.53 (ttd, J=9.9, 3.8, 1.7 Hz, 2H), 1.92-1.52 (m, 10H), 1.34-0.91 (m, 10H).

¹³C NMR (75 MHz, CDCl₃) δ 53.1, 34.4, 26.3, 25.4.

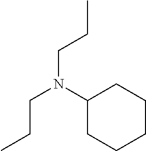

### N-hexylcyclohexanamine (6aj)

The product was prepared using procedure B and isolated in 78% yield (0.721 g, 4.69 mmol) as a colorless liquid after purification via column chromatography on silica gel using hexane the eluent.

¹H NMR (300 MHz, CDCl₃) δ 2.42 (tt, J=10.6, 3.9 Hz, 1H), 2.07-1.07 (m, 24H).

¹³C NMR (75 MHz, CDCl₃) δ 60.3, 36.9, 32.1, 29.3, 27.9, 25.8, 24.3.

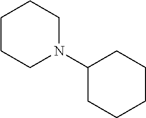 [SMILES: C1CCC(N2CCCCC2)CC1]

### N-hexylcyclohexanamine (6ak)

The product was prepared using procedure D for 18h and isolated in 72% yield (0.856 g, 4.32 mmol) as a colorless liquid after purification via column chromatography on silica gel using hexane the eluent.

¹H NMR (300 MHz, CDCl₃) δ 2.61-2.43 (m, 4H), 2.22 (dt, J=5.8, 2.6 Hz, 1H), 1.90-0.81 (m, 16H).

¹³C NMR (75 MHz, CDCl₃) δ 64.4, 53.1, 50.1, 34.5, 28.9, 26.6, 26.6, 26.3, 25.4, 25.0.

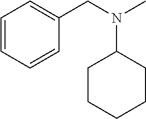

### N-benzyl-N-methylcyclohexanamine (6al)

The product was prepared using procedure B and isolated in 82% yield (0.998 g, 4.92 mmol) as a colorless liquid after purification via column chromatography on silica gel using hexane the eluent.

¹H NMR (300 MHz, CDCl₃) δ 7.65-6.93 (m, 5H), 3.57 (s, 2H), 2.09-1.75 (m, 1H), 2.19 (s, 3H), 1.64 (dtd, J=11.6, 3.5, 2.1 Hz, 4H), 1.43-1.02 (m, 6H).

¹³C NMR (75 MHz, CDCl₃) δ 140.3, 128.7, 128.1, 126.6, 62.6, 57.9, 37.8, 28.8, 26.6, 26.2.

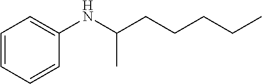

### N-(heptan-2-yl)aniline (6 cm)

The product was prepared using procedure A and isolated in 91% yield (1.050 g, 5.46 mmol) as a brown oil after purification via column chromatography on silica gel using hexane the eluent.

¹H NMR (300 MHz, CDCl₃) δ 7.29-7.10 (m, 2H), 6.73-6.53 (m, 3H), 3.47 (m, 2H), 1.64-1.38 (m, 1H), 1.44-1.30 (m, 3H), 1.35-1.23 (m, 4H), 1.19 (dd, J=6.2, 1.4 Hz, 3H), 0.96-0.85 (m, 3H).

¹³C NMR (75 MHz, CDCl₃) δ 147.6, 129.2, 116.7, 113.0, 48.5, 37.3, 32.0, 26.0, 22.8, 20.9, 14.2.

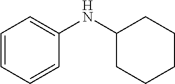 [SMILES: c1ccc(NC2CCCCC2)cc1]

### N-cyclohexylaniline (6am)

The product was prepared using procedure A and isolated in 89% yield (0.934 g, 5.34 mmol) as a yellow oil after purification via column chromatography on silica gel using hexane the eluent.

¹H NMR (300 MHz, CDCl₃) δ 7.29-7.10 (m, 2H), 6.75-6.62 (m, 1H), 6.67-6.52 (m, 2H), 3.56-3.50 (m, 1H), 3.28 (tt, J=10.1, 3.8 Hz, 1H), 2.16-2.01 (m, 2H), 1.87-1.60 (m, 3H), 1.50-1.08 (m, 5H).

¹³C NMR (75 MHz, CDCl₃) δ 147.3, 129.2, 116.8, 113.1, 51.7, 33.6, 26.1, 25.2.

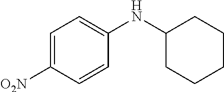 [SMILES: O=[N+]([O-])c1ccc(NC2CCCCC2)cc1]

### N-cyclohexyl-4-nitroaniline (6ao)

The product was prepared using procedure B and isolated in 71% yield (0.937 g, 4.26 mmol) as a yellow solid after purification via column chromatography on silica gel using hexane/EtOAc 95:5 as the eluent.

¹H NMR (300 MHz, CDCl₃) δ 8.06 (d, J=9.2 Hz, 2H), 6.49 (d, J=9.2 Hz, 2H), 4.43 (s, 2H), 2.12-1.98 (m, 3H), 1.86-1.60 (m, 5H), 1.50-1.13 (m, 9H).

¹³C NMR (75 MHz, CDCl₃) δ 152.4, 137.2, 126.5, 111.1, 51.6, 32.9, 25.7, 24.9.

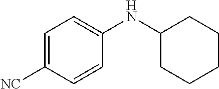 [SMILES: N#Cc1ccc(NC2CCCCC2)cc1]

### N-cyclohexyl-4-cyanoaniline (6an)

The product was prepared using procedure B and isolated in 78% yield (0.936 g, 4.68 mmol) as a yellow solid after purification via column chromatography on silica gel using hexane/EtOAc 95:5 as the eluent.

¹H NMR (300 MHz, CDCl₃) δ 7.17 (tdd, J=7.5, 1.2, 0.6 Hz, 1H), 6.92-6.82 (m, 1H), 6.80-6.69 (m, 2H), 3.84 (s, 1H), 3.21 (tt, J=10.1, 3.8 Hz, 1H), 2.09-1.94 (m, 2H), 1.88-1.59 (m, 3H), 1.47-1.06 (m, 5H).

¹³C NMR (75 MHz, CDCl₃) δ 147.5, 129.8, 119.9, 119.6, 117.4, 114.9, 112.8, 51.5, 33.1, 25.9, 25.0.

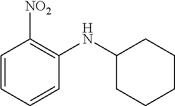 [SMILES: O=[N+]([O-])c1ccccc1NC1CCCCC1]

### N-cyclohexyl-2-nitroaniline (6ap)

The product was prepared using procedure C for 18h and isolated in 82% yield (0.541 g, 2.46 mmol) as a pale yellow liquid after purification via column chromatography on silica gel using hexane/EtOAc (1:1) as an eluent.

¹H NMR (300 MHz, CDCl₃) δ 8.15 (td, J=7.1, 3.3 Hz, 2H), 7.39 (ddd, J=8.3, 6.6, 1.4 Hz, 1H), 6.59 (ddt, J=8.8, 6.9, 1.0 Hz, 1H), 3.51 (d, J=8.9 Hz, 1H), 2.12-2.01 (m, 2H), 1.71-1.61 (m, 1H), 1.53-1.22 (m, 5H).

¹³C NMR (75 MHz, CDCl₃) δ 144.7, 135.9, 131.4, 127.0, 114.7, 114.1, 51.0, 32.8, 25.7, 24.7.

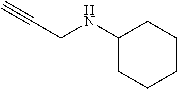

### N-(prop-2-yn-1-yl)cyclohexanamine (6av)

The product was prepared using procedure A and isolated in 92% yield (0.756 g, 5.52 mmol) as a brown liquid after purification via column chromatography on silica gel using hexane/EtOAc 95:5 as the eluent.

¹H NMR (300 MHz, CDCl₃) δ 3.41 (dd, J=2.4, 0.8 Hz, 2H), 2.69-2.43 (m, 1H), 2.16 (td, J=2.4, 0.8 Hz, 1H), 1.94-1.49 (m, 4H), 1.35-0.92 (m, 5H).

¹³C NMR (75 MHz, CDCl₃) δ 82.5, 70.9, 54.9, 35.2, 33.1, 26.2, 24.9.

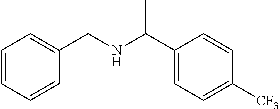 [SMILES: CC(NCc1ccccc1)c1ccc(C(F)(F)F)cc1]

### N-benzyl-1-phenylethan-1-amine (6ga)

The product was prepared using procedure D and isolated in 99% yield (0.626 g, 2.97 mmol) as a colorless liquid after purification via column chromatography on silica gel using hexane/EtOAc (9:1) as an eluent.

¹H NMR (300 MHz, CDCl₃) δ 7.48-7.21 (m, 10H), 3.86 (q, J=6.6 Hz, 1H), 3.76-3.58 (m, 2H), 1.61 (s, 1H), 1.41 (dd, J=6.6, 0.5 Hz, 3H).

¹³C NMR (75 MHz, CDCl₃) δ 145.5, 140.6, 128.4, 128.3, 128.1, 126.9, 126.8, 126.7, 57.6, 51.8, 24.7.

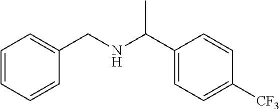 [SMILES: CC(NCc1ccccc1)c1ccc(C(F)(F)F)cc1]

### 1-phenyl-N-(4-(trifluoromethyl)benzyl)ethan-1-amine (6ia)

The product was prepared using procedure D and isolated in 82% yield (0.686 g, 2.46 mmol) as a colorless liquid after purification via column chromatography on silica gel using hexane/EtOAc (9:1) as an eluent.

¹H NMR (300 MHz, CDCl₃) δ 7.62 (d, J=8.1 Hz, 2H), 7.51 (d, J=8.1 Hz, 2H), 7.42-7.20 (m, 5H), 3.90 (q, J=6.5 Hz, 1H), 3.72-3.55 (m, 2H), 1.64 (s, 1H), 1.38 (dd, J=6.6, 1.4 Hz, 3H).

¹³C NMR (75 MHz, CDCl₃) δ 149.7, 140.2, 128.4, 128.0, 127.0, 126.9, 125.4, 125.4, 57.3, 51.8, 24.7.

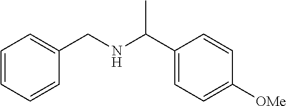 [SMILES: COc1ccc(C(C)NCc2ccccc2)cc1]

### N-(4-methoxybenzyl)-1-phenylethan-1-amine (6ha)

The product was prepared using procedure D and isolated in 99% yield (0.715 g, 2.97 mmol) as a colorless liquid after purification via column chromatography on silica gel using hexane/EtOAc (9:1) as an eluent.

¹H NMR (300 MHz, CDCl₃) δ 7.61-7.12 (m, 7H), 7.02-6.87 (m, 2H), 3.93-3.76 (m, 2H), 3.84 (s, 3H), 3.75-3.56 (m, 2H), 1.40 (d, J=6.6 Hz, 3H).

¹³C NMR (75 MHz, CDCl₃) δ 158.5, 140.7, 137.6, 128.3, 128.1, 127.7, 126.8, 113.8, 56.9, 55.3, 51.7, 24.7.

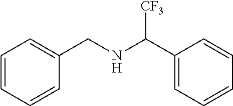 [SMILES: FC(F)(F)C(NCc1ccccc1)c1ccccc1]

### N-benzyl-2,2,2-trifluoro-1-phenylethan-1-amine (6ja)

The product was prepared using procedure D and isolated in 72% yield (0.552 g, 2.16 mmol) as a pale yellow liquid after purification via column chromatography on silica gel using hexane/EtOAc (1:1) as an eluent.

¹H NMR (300 MHz, CDCl₃) δ 7.49-7.35 (m, 1H), 4.99 (q, J=6.8 Hz, OH), 3.43 (s, OH), 3.14 (s, OH).

¹³C NMR (75 MHz, CDCl₃) δ 193.10, 166.10, 134.45, 129.21, 128.61, 128.55, 128.40, 127.44, 73.12, 72.70, 72.28, 71.86, 50.30.

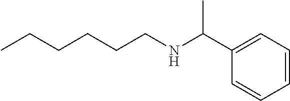

### N-(1-phenylethyl)hexan-1-amine (6gh)

The product was prepared using procedure D and isolated in 87% yield (0.535 g, 2.61 mmol) as a pale blue liquid after purification via column chromatography on silica gel using hexane/EtOAc (9:1) as an eluent.

¹H NMR (300 MHz, CDCl₃) δ 7.58-7.01 (m, 5H), 3.75 (q, J=6.6 Hz, 1H), 2.59-2.34 (m, 2H), 1.57-1.17 (m, 14H), 0.88 (dt, J=9.2, 4.5 Hz, 3H).

¹³C NMR (75 MHz, CDCl₃) δ 145.8, 128.3, 126.7, 126.5, 58.5, 48.0, 31.9, 30.4, 27.2, 24.5, 22.7, 14.2.

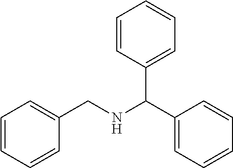 [SMILES: c1ccc(CNC(c2ccccc2)c2ccccc2)cc1]

### N-benzyl-1,1-diphenylmethanamine (6ka)

The product was prepared using procedure D and isolated in 90% yield (0.737 g, 2.7 mmol) as a colorless liquid after purification via column chromatography on silica gel using hexane/EtOAc (9:1) as an eluent.

¹H NMR (300 MHz, CDCl₃) δ 7.83-6.87 (m, 15H), 4.94 (s, 1H), 3.83 (s, 2H), 1.94 (s, 1H).

¹³C NMR (75 MHz, CDCl₃) δ 143.9, 140.4, 128.5, 128.4, 128.2, 127.4, 127.0, 126.9, 66.6, 52.0.

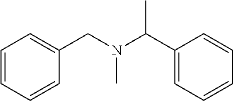 [SMILES: CC(c1ccccc1)N(C)Cc1ccccc1]

### N-benzyl-N-methyl-1-phenylethan-1-amine (6gl)

The product was prepared using procedure D and isolated in 96% yield (0.648 g, 2.88 mmol) as a colorless liquid after purification via column chromatography on silica gel using hexane/EtOAc (9:1) as an eluent.

¹H NMR (300 MHz, CDCl₃) δ 7.51-7.20 (m, 10H), 3.82-3.51 (m, 2H), 3.35 (d, J=13.3 Hz, 1H), 2.19 (s, 3H), 1.47 (dt, J=6.7, 0.8 Hz, 3H).

¹³C NMR (75 MHz, CDCl₃) δ 144.1, 140.0, 128.7, 128.1, 127.6, 126.8, 126.7, 63.3, 59.0, 38.5, 18.6.

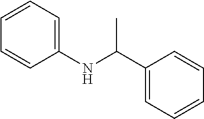

### N-(1-phenylethyl)aniline (6 gm)

The product was prepared using procedure D and isolated in 95% yield (0.561 g, 2.85 mmol) as a colorless liquid after purification via column chromatography on silica gel using hexane/EtOAc (9:1) as an eluent.

¹H NMR (300 MHz, CDCl₃) δ 7.47-7.33 (m, 4H), 7.33-7.23 (m, 1H), 7.15 (t, J=7.7 Hz, 2H), 6.70 (t, J=7.3 Hz, 1H), 6.57 (d, J=7.9 Hz, 2H), 4.54 (q, J=6.7 Hz, 1H), 4.07 (s, 1H), 1.56 (d, J=6.9 Hz, 3H)

¹³C NMR (75 MHz, CDCl₃) δ 147.2, 145.2, 129.1, 128.6, 126.8, 125.8, 117.2, 113.3, 53.5, 25.2.

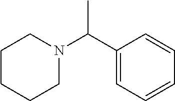

### 1-(1-phenylethyl)piperidine (6gk)

The product was prepared using procedure D and isolated in 86% yield (0.487 g, 2.58 mmol) as a pale yellow liquid after purification via column chromatography on silica gel using hexane/EtOAc (8:2) as an eluent.

¹H NMR (300 MHz, CDCl₃) δ 7.38-7.17 (m, 5H), 3.47-3.34 (m, 1H), 2.39 (tp, J=11.5, 5.2 Hz, 4H), 1.57 (dq, J=10.7, 5.5 Hz, 6H), 1.49-1.35 (m, 3H).

¹³C NMR (75 MHz, CDCl₃) δ 143.8, 127.9, 127.7, 126.6, 65.3, 51.6, 26.4, 24.8, 19.6.

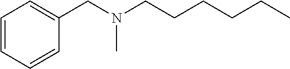

### N-benzyl-N-methylhexan-1-amine (7bl)

The product was prepared using procedure C and isolated in 88% yield (0.541 g, 2.64 mmol) as a colorless liquid after purification via column chromatography on silica gel using hexane the eluent.

¹H NMR (300 MHz, CDCl₃) δ 7.38-7.18 (m, 5H), 3.48 (s, 2H), 2.42-2.31 (m, 2H), 2.19 (s, 3H), 1.51 (qd, J=6.7, 3.6 Hz, 2H), 1.41-1.20 (m, 6H), 0.95-0.84 (m, 3H).

¹³C NMR (75 MHz, CDCl₃) δ 139.2, 129.0, 128.1, 126.7, 62.4, 57.7, 42.4, 31.9, 27.5, 27.3, 22.8, 14.2.

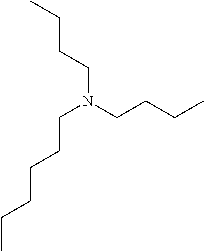

### N,N-dibutylhexan-1-amine (7br)

The product was prepared using procedure C and isolated in 76% yield (0.485 g, 2.28 mmol) as a colorless liquid after purification via column chromatography on silica gel using hexane the eluent. HRMS (CI) m/z: [M+H]+ calcd. for C₁₄H3₂N 214.2535, found 214.2535.

¹H NMR (300 MHz, CDCl₃) δ 2.36 (tt, J=7.9, 2.3 Hz, 6H), 1.68-1.11 (m, 16H), 0.89 (t, J=7.1 Hz, 9H).

¹³C NMR (75 MHz, CDCl₃) δ 54.3, 54.0, 32.0, 29.3, 27.4, 27.1, 22.8, 20.9, 14.2.

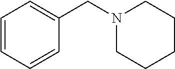 [SMILES: c1ccc(CN2CCCCC2)cc1]

### Benzylpiperidine (7ak)

The product was prepared using procedure C and isolated in 97% yield (0.509 g, 2.91 mmol) as a clear yellow liquid after purification via column chromatography on silica gel using hexane/EtOAc (95:5) as an eluent.

¹H NMR (300 MHz, Chloroform-d) δ 7.40-7.23 (m, 5H), 3.51 (s, 2H), 2.42 (t, J=5.3 Hz, 4H), 1.71-1.58 (m, 4H), 1.48 (td, J=6.1, 3.2 Hz, 2H).

¹³C{1H} NMR (75 MHz, CDCl₃) δ 138.6, 129.2, 128.0, 126.8, 64.0, 54.6, 26.2, 24.6.

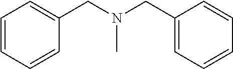 [SMILES: CN(Cc1ccccc1)Cc1ccccc1]

### N-benzyl-N-methyl-1-phenylmethanamine (7al)

The product was prepared using procedure C and isolated in 98% yield (0.620 g, 2.94 mmol) as a colorless liquid after purification via column chromatography on silica gel using hexane the eluent.

¹H NMR (300 MHz, CDCl₃) δ 7.67-7.00 (m, 10H), 3.58 (s, 4H), 2.27-2.21 (m, 3H).

¹³C NMR (75 MHz, CDCl₃) δ 139.3, 128.9, 128.2, 126.9, 61.9, 42.4.

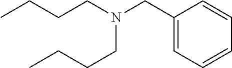 [SMILES: CCCCN(CCCC)Cc1ccccc1]

### N-benzyl-N-butylbutan-1-amine (7ar)

The product was prepared using procedure C and isolated in 99% yield (0.620 g, 2.94 mmol) as a colorless liquid after purification via column chromatography on silica gel using hexane the eluent.

¹H NMR (300 MHz, CDCl₃) δ 7.67-7.00 (m, 5H), 3.58 (s, 2H), 2.27-2.21 (m, 4H), 1.5-1.3 (m, 8H), 0.9 (t, J=6.5 Hz, 6H).

¹³C NMR (75 MHz, CDCl₃) δ 139.3, 128.7, 127.9, 126.5, 58.7, 53.6, 29.4, 20.8, 14.2.

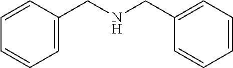 [SMILES: c1ccc(CNCc2ccccc2)cc1]

### Dibenzyl amine (7aa)

The product was prepared using procedure C and isolated in 54% yield (0.319 g, 1.54 mmol) as a colorless liquid after purification via column chromatography on silica gel using hexane/EtOAc (95:5) as an eluent.

¹H NMR (300 MHz, Chloroform-d) δ 7.42-7.25 (m, 10H), 3.85 (s, 4H), 2.09 (s, 1H).

¹³C{1H} NMR (75 MHz, CDCl₃) δ 140.0, 128.4, 128.2, 127.0, 53.1.

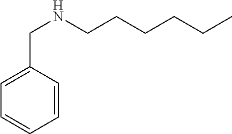

### N-benzylhexan-1-amine (7ca)

The product was prepared using procedure C and isolated in 70% yield (0.401 g, 2.1 mmol) as a colorless liquid after purification via column chromatography on silica gel using hexane/EtOAc (95:5) as an eluent.

¹H NMR (300 MHz, CDCl₃) δ 7.33-7.17 (m, 5H), 3.8 (s, 2H), 2.63 (t, J=7.2 Hz, 2H), 1.62-1.39 (m, 2H), 1.36-1.28 (m, 6H), 0.92-0.83 (m, 3H).

¹³C NMR (75 MHz, CDCl₃) δ 140.5, 128.3, 128.0, 126.8, 54.2, 49.6, 31.9, 30.2, 27.2, 22.8, 14.2.

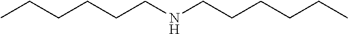

### N-benzylhexan-1-amine (7ch)

The product was prepared using procedure C and isolated in 77% yield (0.427 g, 2.31 mmol) as a colorless liquid after purification via column chromatography on silica gel using hexane/EtOAc (95:5) as an eluent.

¹H NMR (300 MHz, CDCl₃) δ 2.55 (t, J=6.9 Hz, 4H), 1.53-1.36 (m, 4H), 1.28 (ddt, J=10.0, 3.9, 2.0 Hz, 12H), 0.95-0.78 (m, 6H).

¹³C NMR (75 MHz, CDCl₃) δ 50.3, 31.9, 30.3, 27.2, 22.7, 14.1.

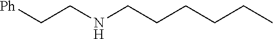

### N-phenethylhexan-1-amine (7cg)

The product was prepared using procedure C and isolated in 72% yield (0.442 g, 2.16 mmol) as a colorless liquid after purification via column chromatography on silica gel using hexane/EtOAc (95:5) as an eluent.

¹H NMR (300 MHz, CDCl₃) δ 7.88-6.98 (m, 5H), 2.97-2.73 (m, 4H), 2.69-2.55 (m, 2H), 1.37-1.18 (m, 8H), 0.95-0.82 (m, 3H).

¹³C NMR (75 MHz, CDCl₃) δ 140.1, 128.6, 128.4, 126.0, 50.0, 36.5, 31.9, 30.2, 27.2, 22.7, 14.2, 1.2.

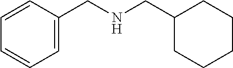 [SMILES: c1ccc(CNCC2CCCCC2)cc1]

### N-benzyl-1-cyclohexylmethanamine (7ba)

The product was prepared using procedure C and isolated in 84% yield (0.511 g, 2.5 mmol) as a pale yellow liquid after purification via column chromatography on silica gel using hexane as an eluent.

¹H NMR (300 MHz, Chloroform-d) δ 7.31 (dd, J=23.7, 4.5 Hz, 5H), 3.80 (s, 2H), 2.50 (d, J=6.7 Hz, 2H), 1.89-1.63 (m, 4H), 1.62-1.46 (m, 1H), 1.42-1.09 (m, 4H), 0.97 (td, J=11.7, 2.9 Hz, 2H).

¹³C{1H} NMR (75 MHz, CDCl₃) δ 140.6, 128.3, 128.1, 128.0, 126.8, 56.3, 54.3, 38.1, 31.9, 31.6, 26.9, 26.3.

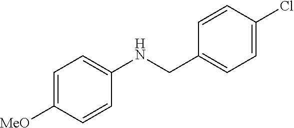 [SMILES: COc1ccc(NCc2ccc(Cl)cc2)cc1]

### N-(4-chlorobenzyl)-4-methoxyaniline (7dq)

The product was prepared using procedure C and isolated in 96% yield (0.714 g, 2.88 mmol) as a yellow liquid after purification via column chromatography on silica gel using hexane/EtOAc (90:10) as an eluent.

¹H NMR (300 MHz, CDCl₃) δ 7.31 (s, 4H), 6.79 (d, J=8.9 Hz, 2H), 6.59 (d, J=8.9 Hz, 2H), 4.27 (s, 2H), 3.75 (s, 3H).

¹³C NMR (75 MHz, CDCl₃) δ 152.2, 142.0, 138.2, 132.7, 128.7, 128.6, 114.9, 114.1, 55.9, 48.6.

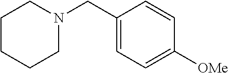 [SMILES: COc1ccc(CN2CCCCC2)cc1]

### 1-(4-methoxybenzyl)piperidine (7dk)

The product was prepared using procedure C and isolated in 94% yield (0.578 g, 2.82 mmol) as a colorless liquid after purification via column chromatography on silica gel using hexane the eluent.

¹H NMR (300 MHz, CDCl₃) δ 7.29-7.17 (m, 2H), 3.79 (d, J=0.5 Hz, 3H), 3.42 (s, 2H), 2.36 (t, J=5.3 Hz, 4H), 1.57 (p, J=5.5 Hz, 4H), 1.42 (hept, J=4.3 Hz, 2H).

¹³C NMR (75 MHz, CDCl₃) δ 158.4, 130.3, 113.4, 63.2, 55.3, 54.4, 26.0, 24.5.

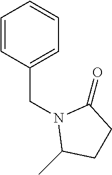

### 1-benzyl-5-methylpyrrolidin-2-one (9aa)

The product was prepared using procedure E and isolated in 92% yield (1.043 g, 5.52 mmol) as a pale yellow liquid.

¹H NMR (300 MHz, CDCl₃) δ 7.37-7.16 (m, 5H), 4.95 (d, J=15.0 Hz, 1H), 3.97 (dd, J=15.0, 1.0 Hz, 1H), 3.51 (dp, J=7.6, 6.2 Hz, 1H), 2.58-2.30 (m, 2H), 2.14 (dddd, J=12.6, 9.4, 7.5, 6.0 Hz, 1H), 1.58 (dddd, J=12.9, 9.5, 7.3, 5.8 Hz, 1H), 1.15 (d, J=6.3 Hz, 3H).

¹³C NMR (75 MHz, CDCl₃) δ 174.8, 136.7, 128.5, 127.9, 127.3, 52.9, 43.9, 30.4, 26.7, 19.7.

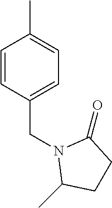

### 5-methyl-1-(4-methylbenzyl)pyrrolidin-2-one (9as)

The product was prepared using procedure E and isolated in 90% yield (1.116 g, 5.5 mmol) as a pale yellow liquid.

¹H NMR (300 MHz, CDCl₃) δ 7.12 (d, 4H), δ 4.94 (d, J=14.9 Hz, 1H), 3.98-3.87 (m, 1H), 3.51 (dt, J=7.6, 6.1 Hz, 1H), 2.59-2.27 (m, 5H), 2.23-2.05 (m, 1H), 1.67-1.49 (m, 1H), 1.16 (dd, J=6.3, 0.6 Hz, 3H).

¹³C NMR (75 MHz, CDCl₃) δ 174.9, 137.0, 133.5, 129.2, 127.9, 52.9, 43.7, 30.4, 26.7, 21.2, 19.7.

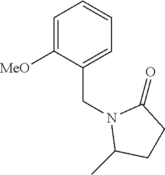 [SMILES: COc1ccccc1CN1C(=O)CCC1C]

### 1-(2-methoxybenzyl)-5-methylpyrrolidin-2-one (9ab)

The product was prepared using procedure E and isolated in 88% yield (1.156 g, 5.28 mmol) as a pale yellow liquid.

¹H NMR (300 MHz, CDCl₃) δ 7.29-7.13 (m, 2H), 6.96-6.81 (m, 2H), 4.81 (d, J=15.3 Hz, 1H), 4.26-4.15 (m, 1H), 3.82 (s, 3H), 3.68-3.45 (m, 1H), 2.58-2.30 (m, 2H), 2.15 (dddd, J=12.7, 9.5, 7.6, 6.2 Hz, 1H), 1.59 (dddd, J=12.7, 9.5, 7.0, 5.5 Hz, 1H), 1.18 (d, J=6.3 Hz, 3H).

¹³C NMR (75 MHz, CDCl₃) δ 175.0, 157.2, 129.4, 128.4, 124.7, 120.5, 110.2, 55.4, 53.4, 38.4, 30.3, 26.8, 19.7.

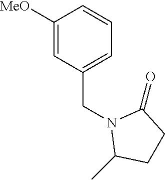 [SMILES: COc1cccc(CN2C(=O)CCC2C)c1]

### 1-(3-methoxybenzyl)-5-methylpyrrolidin-2-one (9ac)

The product was prepared using procedure E and isolated in 83% yield (1.09 g, 4.98 mmol) as a pale yellow liquid.

¹H NMR (300 MHz, CDCl₃) δ 7.29-7.15 (m, 1H), 6.83-6.71 (m, 3H), 4.91 (d, J=15.0 Hz, 1H), 3.93 (d, J=15.0 Hz, 1H), 3.76 (d, J=0.7 Hz, 3H), 3.61-3.39 (m, 1H), 2.57-2.29 (m, 2H), 2.22-2.04 (m, 1H), 1.66-1.48 (m, 1H), 1.14 (dd, J=6.3, 0.7 Hz, 3H).

¹³C NMR (75 MHz, CDCl₃) δ 174.9, 159.7, 138.2, 129.5, 120.2, 113.5, 112.7, 55.3, 52.9, 43.9, 30.3, 26.7, 19.7.

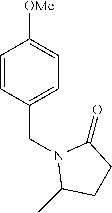 [SMILES: COc1ccc(CN2C(=O)CCC2C)cc1]

### 1-(4-methoxybenzyl)-5-methylpyrrolidin-2-one (9ad)

The product was prepared using procedure E and isolated in 95% yield (1.25 g, 5.7 mmol) as a pale yellow liquid.

¹H NMR (300 MHz, CDCl₃) δ 7.20-7.09 (m, 2H), 6.82 (d, J=8.6 Hz, 2H), 4.89 (d, J=14.8 Hz, 1H), 3.89 (d, J=14.8 Hz, 1H), 3.77 (d, J=0.6 Hz, 3H), 3.57-3.41 (m, 1H), 2.56-2.28 (m, 2H), 2.20-2.03 (m, 1H), 1.56 (dddd, J=12.9, 9.6, 7.3, 5.8 Hz, 1H), 1.14 (dd, J=6.3, 0.6 Hz, 3H).

¹³C NMR (75 MHz, CDCl₃) δ 174.8, 158.8, 129.2, 128.7, 113.9, 55.3, 52.7, 43.3, 30.4, 26.7, 19.7.

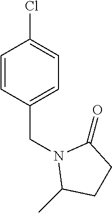

### 1-(4-chlorobenzyl)-5-methylpyrrolidin-2-one (9af)

The product was prepared using procedure E and isolated in 92% yield (1.23 g, 5.5 mmol) as a pale yellow.

¹H NMR (300 MHz, CDCl₃) δ 7.35-7.22 (m, 2H), 7.21-7.11 (m, 2H), 4.85 (d, J=15.1 Hz, 1H), 3.99 (d, J=15.1 Hz, 1H), 3.50 (dp, J=7.5, 6.2 Hz, 1H), 2.57-2.29 (m, 2H), 2.15 (dddd, J=13.1, 9.3, 7.5, 5.9 Hz, 1H), 1.59 (dddd, J=13.0, 9.5, 7.4, 5.9 Hz, 1H), 1.14 (d, J=6.3 Hz, 3H).

¹³C NMR (75 MHz, CDCl₃) δ 174.9, 135.4, 133.1, 129.2, 128.7, 53.0, 43.4, 30.3, 26.8, 19.8.

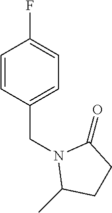

### 1-(4-fluorobenzyl)-5-methylpyrrolidin-2-one (9ae)

The product was prepared using procedure E and isolated in 78% yield (0.968 g, 4.68 mmol) as a colorless liquid.

¹H NMR (300 MHz, CDCl₃) δ 7.31-7.13 (m, 2H), 7.11-6.89 (m, 2H), 4.86 (d, J=15.0 Hz, 1H), 3.98 (d, J=15.0 Hz, 1H), 3.51 (dq, J=7.6, 6.2 Hz, 1H), 2.70-2.03 (m, 3H), 1.69-1.51 (m, 1H), 1.15 (dd, J=6.3, 0.6 Hz, 3H).

¹³C NMR (75 MHz, CDCl₃) δ 175.5, 163.6, 132.2, 129.6, 129.5, 115.6, 115.3, 53.2, 43.4, 30.2, 26.7, 19.7.

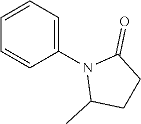

### 5-methyl-1-phenylpyrrolidin-2-one(9am)

The product was prepared using procedure E for 6 h and isolated in 91% yield (0.956 g, 5.46 mmol) as a colourless liquid.

¹H NMR (300 MHz, CDCl₃) δ 7.45-7.31 (m, 3H), 7.31-7.11 (m, 2H), 4.29 (dp, J=7.2, 6.1 Hz, 1H), 2.73-2.26 (m, 3H), 1.83-1.65 (m, 1H), 1.19 (dd, J=6.3, 0.5 Hz, 3H).

¹³C NMR (75 MHz, CDCl₃) δ 174.2, 137.4, 128.9, 125.8, 124.0, 55.8, 53.23, 31.4, 26.8, 20.3.

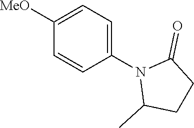

### 1-(4-methoxyphenyl)-5-methylpyrrolidin-2-one (9aq)

The product was prepared using procedure E and isolated in 98% yield (1.206 g, 5.88 mmol) as a colourless liquid.

¹H NMR (300 MHz, CDCl₃) δ 7.29-7.17 (m, 2H), 7.01-6.87 (m, 2H), 4.18 (dt, J=7.6, 6.0 Hz, 1H), 3.83-3.77 (s, 3H), 2.71-2.53 (m, 2H), 2.58-2.24 (m, 1H), 1.90-1.63 (m, 1H), 1.21-1.13 (m, 3H).

¹³C NMR (75 MHz, CDCl₃) δ 174.3, 157.6, 126.1, 114.3, 56.3, 55.5, 31.2, 26.9, 20.4.

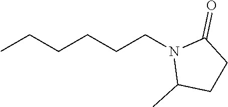

### 1-hexyl-5-methylpyrrolidin-2-one (9ah)

The product was prepared using procedure E and isolated in 84% yield (0.992 g, 5.04 mmol) as a colorless liquid.

¹H NMR (300 MHz, CDCl₃) δ 3.75-3.44 (m, 2H), 2.88 (dddd, J=13.9, 8.9, 5.8, 3.3 Hz, 1H), 2.49-2.06 (m, 3H), 1.63-1.34 (m, 1H), 1.26 (d, J=4.4 Hz, 2H), 1.25-1.11 (m, 11H), 0.89-0.78 (m, 3H).

¹³C NMR (75 MHz, CDCl₃) δ 174.8, 53.4, 40.1, 31.6, 30.4, 27.4, 26.8, 26.7, 22.6, 19.8, 14.1.

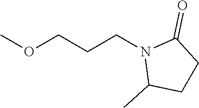

### 1-(3-methoxypropyl)-5-methylpyrrolidin-2-one(9at)

The product was prepared using procedure E and isolated in 76% yield (0.779 g, 4.56 mmol) as a pale-yellow liquid.

¹H NMR (300 MHz, CDCl₃) δ 3.83-3.49 (m, 2H), 3.34 (td, J=6.2, 1.7 Hz, 2H), 3.27 (d, J=2.2 Hz, 3H), 3.11-2.95 (m, 1H), 2.51-2.08 (m, 3H), 1.79-1.47 (m, 3H), 1.19 (dd, J=6.4, 1.7 Hz, 3H).

¹³C NMR (75 MHz, CDCl₃) δ 175.5, 70.2, 58.7, 54.1, 37.8, 30.2, 27.7, 26.9, 19.8.

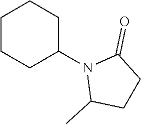

### 1-cyclohexyl-5-methylpyrrolidin-2-one (9ai)

The product was prepared using procedure E and isolated in 40% yield (0.434 g, 2.4 mmol) and in 89% yield (0.96 g, 5.3 mmol) when prepared using procedure D in 48h. as a colourless liquid.

¹H NMR (300 MHz, CDCl₃) δ 3.94-3.61 (m, 2H), 2.45 (dt, J=16.4, 8.9 Hz, 1H), 2.33-1.93 (m, 2H), 1.87-1.37 (m, 6H), 1.31 (ddt, J=12.5, 6.5, 3.8 Hz, 2H), 1.31-1.10 (m, 4H), 1.07 (t, J=3.5 Hz, 2H).

¹³C NMR (75 MHz, CDCl₃) δ 174.4, 53.0, 52.6, 31.9, 30.5, 30.2, 27.6, 26.1, 26.0, 25.7, 22.5.

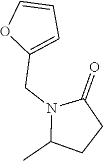

### 1-(furan-2-ylmethyl)-5-methylpyrrolidin-2-one(9au)

The product was prepared using procedure E and isolated in 83% yield (0.891g, 4.98 mmol) as a pale-yellow liquid.

¹H NMR (300 MHz, CDCl₃) δ 6.27 (dd, J=3.2, 1.9 Hz, 2H), 6.19 (dd, J=3.2, 0.8 Hz, 1H), 4.83 (d, J=15.6 Hz, 1H), 4.02 (d, J=15.6 Hz, 1H), 3.64-3.48 (m, 1H), 2.51-2.21 (m, 2H), 2.13 (dddd, J=12.6, 9.3, 7.4, 5.9 Hz, 1H), 1.54 (dddd, J=12.6, 9.6, 7.4, 5.9 Hz, 1H), 1.19 (d, J=6.3 Hz, 3H).

¹³C NMR (75 MHz, CDCl₃) δ 174.6, 150.2, 142.1, 110.3, 108.1, 53.3, 36.8, 30.2, 26.7, 19.7.

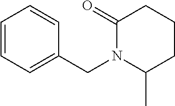

### 1-benzyl-6-methylpiperidin-2-one (9ba)

The product was prepared using procedure E and isolated in 90% yield (1.096 g, 5.4 mmol) as a pale-yellow liquid.

¹H NMR (300 MHz, CDCl₃) δ 7.27 (ddd, J=4.9, 1.9, 1.0 Hz, 1H), 7.17-6.95 (m, 3H), 6.90 (dd, J=8.8, 0.8 Hz, 2H), 5.08 (d, J=14.6 Hz, 1H), 4.36 (dd, J=7.8, 5.9 Hz, 1H), 3.83 (d, J=0.8 Hz, 3H), 3.51-3.40 (m, 1H), 2.85-2.57 (m, 1H), 2.57-2.24 (m, 2H), 1.88 (dddd, J=13.1, 10.3, 6.5, 5.6 Hz, 1H).

¹³C NMR (75 MHz, CDCl₃) δ 175.1, 159.3, 136.3, 132.5, 128.4, 127.9, 127.4, 114.3, 60.8, 55.4, 44.2, 30.5, 28.5.

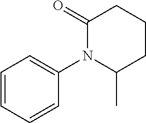

### 1-benzyl-6-methylpiperidin-2-one (9bm)

The product was prepared using procedure E and isolated in 87% yield (0.986 g, 5.22 mmol) as a brown oil.

¹H NMR (300 MHz, CDCl₃) δ 7.39 (t, J=7.6 Hz, 2H), 7.42-7.21 (m, 1H), 7.20-7.10 (m, 2H), 3.92 (q, J=6.0 Hz, 1H), 2.68-2.45 (m, 2H), 2.02 (s, 2H), 2.20-1.57 (m, 2H), 1.08 (d, J=6.4 Hz, 3H).

¹³C NMR (75 MHz, CDCl₃) δ 170.9, 141.1, 129.1, 127.9, 127.2, 55.9, 32.6, 30.7, 20.9, 18.2.

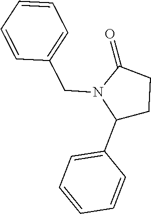 [SMILES: O=C1CCC(c2ccccc2)N1Cc1ccccc1]

### 1-benzyl-5-phenylpyrrolidin-2-one (9ca)

The product was prepared using procedure F and isolated in 82% yield (0.617 g, 2.46 mmol) as a yellow liquid.

¹H NMR (300 MHz, CDCl₃) δ 7.94-6.83 (m, 10H), 5.12 (d, J=14.6 Hz, 1H), 4.41 (dd, J=8.0, 5.6 Hz, 1H), 3.48 (dd, J=14.6, 1.1 Hz, 1H), 2.74-2.31 (m, 3H), 2.00-1.81 (m, 1H).

¹³C NMR (75 MHz, CDCl₃) δ 175.2, 140.7, 136.3, 128.9, 128.5, 128.0, 127.5, 126.7, 61.3, 44.4, 30.4, 28.3.

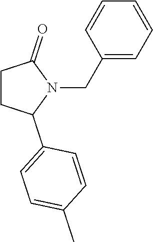 [SMILES: Cc1ccc(C2CCC(=O)N2Cc2ccccc2)cc1]

### 1-benzyl-5-(p-tolyl)pyrrolidin-2-one (9da)

The product was prepared using procedure F and isolated in 87% yield (0.691g, 2.61 mmol) as a yellow liquid.

¹H NMR (300 MHz, CDCl₃) δ 7.37-6.67 (m, 10H), 5.11 (d, J=14.5 Hz, 1H), 4.37 (dd, J=7.9, 5.6 Hz, 1H), 3.46 (dd, J=14.5, 1.1 Hz, 1H), 2.72-2.55 (m, 1H), 2.59-2.24 (m, 5H), 1.96-1.79 (m, 1H).

¹³C NMR (75 MHz, CDCl₃) δ 175.2, 137.6, 136.3, 129.6, 128.4, 127.4, 126.6, 61.0, 44.3, 30.5, 28.4, 21.3.

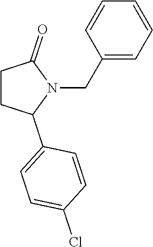 [SMILES: O=C1CCC(c2ccc(Cl)cc2)N1Cc1ccccc1]

### 1-benzyl-5-(p-chloro)pyrrolidin-2-one (9ea)

The product was prepared using procedure F and isolated in 80% yield (0.684g, 2.4 mmol) as a pale-yellow liquid.

¹H NMR (300 MHz, CDCl₃) δ 7.40-7.10 (m, 6H), 7.10-7.00 (m, 3H), 5.11 (d, J=14.6 Hz, 1H), 4.37 (dd, J=8.0, 5.8 Hz, 1H), 3.46 (dd, J=14.6, 1.1 Hz, 1H), 2.72-2.30 (m, 3H), 1.95-1.75 (m, 1H).

¹³C NMR (75 MHz, CDCl₃) δ 175.1, 139.2, 136.0, 133.8, 129.1, 128.5, 128.4, 128.0, 127.5, 60.7, 44.4, 30.3, 28.3.

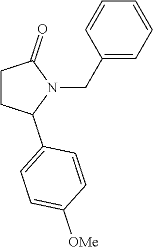 [SMILES: COc1ccc(C2CCC(=O)N2Cc2ccccc2)cc1]

### 1-benzyl-5-(p-methoxy) pyrrolidin-2-one (9fa)

The product was prepared using procedure F and isolated in 83% yield (0.699 g, 2.49 mmol) as a colorless liquid.

¹H NMR (300 MHz, CDCl₃) δ 7.41-6.67 (m, 9H), 5.08 (d, J=14.6 Hz, 1H), 4.36 (dd, J=7.8, 5.9 Hz, 1H), 3.83 (d, J=0.8 Hz, 3H), 3.46 (dd, J=14.5, 1.1 Hz, 1H), 2.85-2.57 (m, 1H), 2.57-2.40 (m, 1H), 2.46-2.24 (m, 1H), 1.88 (dddd, J=13.1, 10.3, 6.5, 5.6 Hz, 1H).

¹³C NMR (75 MHz, CDCl₃) δ 175.1, 159.3, 136.3, 132.5, 128.4, 127.9, 127.4, 114.3, 60.8, 55.4, 44.2, 30.5, 28.4.

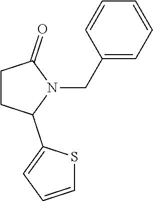 [SMILES: O=C1CCC(c2cccs2)N1Cc1ccccc1]

### 1-benzyl-5-(thiophen-2-yl)pyrrolidin-2-one (9ga)

The product was prepared using procedure F and isolated in 92% yield (0.709 g, 2.76 mmol) as a pale-yellow liquid.

¹H NMR (300 MHz, CDCl₃) δ 7.42-6.49 (m, 8H), 5.08 (d, J=14.7 Hz, 1H), 4.71 (dd, J=7.4, 5.3 Hz, 1H), 3.59 (dd, J=14.7, 1.2 Hz, 1H), 2.85-2.59 (m, 1H), 2.58-2.34 (m, 2H), 2.18-1.96 (m, 1H).

¹³C NMR (75 MHz, CDCl₃) δ 174.4, 144.5, 136.3, 128.5, 128.4, 127.5, 126.7, 125.9, 125.5, 56.8, 44.3, 30.3, 28.9.

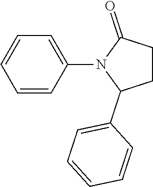 [SMILES: O=C1CCC(c2ccccc2)N1c1ccccc1]

### 1,5-diphenylpyrrolidin-2-one (9 cm)

The product was prepared using procedure F and isolated in 76% yield (0.540 g, 2.28 mmol) as a pale-yellow liquid.

¹H NMR (300 MHz, CDCl₃) δ 7.53-7.33 (m, 2H), 7.36-7.11 (m, 7H), 7.13-7.00 (m, 1H), 5.25 (dd, J=7.2, 4.5 Hz, 1H), 2.87-2.48 (m, 3H), 2.11-1.92 (m, 1H).

¹³C NMR (75 MHz, CDCl₃) δ 174.8, 141.1, 128.9, 128.6, 127.7, 125.8, 124.9, 122.1, 64.0, 31.3, 29.3.

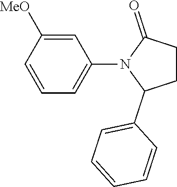 [SMILES: COc1cccc(N2C(=O)CCC2c2ccccc2)c1]

### 1-(4-methoxybenzyl)-5-phenylpyrrolidin-2-one (9cd)

The product was prepared using procedure F and isolated in 89% yield (0.750 g, 2.67 mmol) as a pale-yellow liquid.

¹H NMR (300 MHz, CDCl₃) δ 7.50-7.27 (m, 4H), 7.14 (dd, J=7.9, 1.7 Hz, 1H), 7.08-6.93 (m, 2H), 6.93-6.72 (m, 2H), 5.05 (d, J=14.4 Hz, 1H), 4.39 (dd, J=8.0, 5.7 Hz, 1H), 3.79 (s, 3H), 3.52-3.36 (m, 1H), 2.72-2.24 (m, 3H), 1.99-1.79 (m, 1H).

¹³C NMR (75 MHz, CDCl₃) δ 175.2, 158.8, 140.7, 129.8, 128.9, 128.3, 128.0, 126.7, 113.8, 61.2, 55.3, 43.8, 30.5, 28.4.

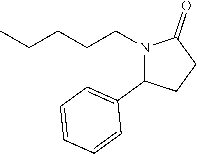 [SMILES: CCCCCN1C(=O)CCC1c1ccccc1]

### 1-pentyl-5-phenylpyrrolidin-2-one (9ch)

The product was prepared using procedure F and isolated in 81% yield (0.561 g, 2.43 mmol) as a white solid.

¹H NMR (300 MHz, CDCl₃) δ 7.43-7.22 (m, 3H), 7.25-7.13 (m, 2H), 4.62 (td, J=5.6, 1.6 Hz, 1H), 3.73-3.57 (m, 1H), 2.65-2.20 (m, 4H), 1.97-1.75 (m, 1H), 1.19 (p, J=5.0 Hz, 6H), 0.94-0.75 (m, 3H).

¹³C NMR (75 MHz, CDCl₃) δ 175.1, 141.1, 128.8, 127.9, 126.4, 62.1, 40.7, 31.5, 30.4, 28.6, 26.9, 26.6, 22.6, 14.1.

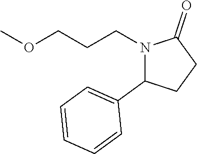 [SMILES: COCCCN1C(=O)CCC1c1ccccc1]

### 1-(3-methoxypropyl)-5-phenylpyrrolidin-2-one (9ct)

The product was prepared using procedure F and isolated in 78% yield (0.545 g, 2.34 mmol) as a pale-yellow liquid.

¹H NMR (300 MHz, CDCl₃) δ 7.31 (dddd, J=13.8, 6.7, 5.9, 1.9 Hz, 3H), 7.16 (dt, J=6.2, 1.7 Hz, 2H), 4.63 (dt, J=8.3, 3.2 Hz, 1H), 3.68 (dtd, J=13.8, 7.6, 1.7 Hz, 1H), 3.38-3.15 (m, 5H), 2.78-2.63 (m, 1H), 2.53-2.30 (m, 3H), 1.96-1.76 (m, 1H), 1.74-1.56 (m, 2H).

¹³C NMR (75 MHz, CDCl₃) δ 175.3, 141.1, 77.5, 77.1, 76.7, 70.4, 62.3, 58.6, 38.3, 30.3, 28.7, 27.3.

HR-MS (ESI) m/z: [M+Na]+ caled for C₁₄H19NO2Na+256.1308, found 256.1313.

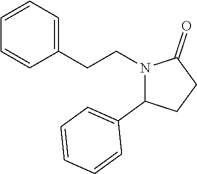 [SMILES: O=C1CCC(c2ccccc2)N1CCc1ccccc1]

### 1-phenethyl-5-phenylpyrrolidin-2-one (9cg)

The product was prepared using procedure F and isolated in 86% yield (0.683 g, 2.58 mmol) as a colorless liquid.

¹H NMR (300 MHz, CDCl₃) δ 7.42-7.04 (m, 10H), 4.35 (dd, J=7.3, 5.6 Hz, 1H), 4.00-3.81 (m, 1H), 2.95-2.24 (m, 6H), 1.94-1.76 (m, 1H).

¹³C NMR (75 MHz, CDCl₃) δ 175.2, 140.9, 138.8, 128.9, 128.6, 128.4, 128.1, 126.6, 126.3, 62.8, 42.3, 33.6, 30.4, 28.7.

HR-MS (ESI) m/z: [M+Na]+ calcd for C₁₈H19NONa+288.1359, found 288.1359.

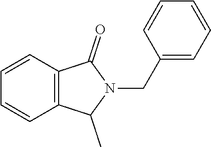 [SMILES: CC1c2ccccc2C(=O)N1Cc1ccccc1]

### 2-benzyl-3-methylisoindolin-1-one (9ha)

The product was prepared using procedure F and isolated in 78% yield (0.554 g, 2.34 mmol) as a pale-yellow liquid.

¹H NMR (300 MHz, CDCl₃) δ 7.93-7.84 (m, 1H), 7.62-7.43 (m, 2H), 7.38-7.20 (m, 6H), 5.34 (d, J=15.2 Hz, 1H), 4.26 (d, J=15.2 Hz, 1H), 1.43 (d, J=6.7 Hz, 3H).

¹³C NMR (75 MHz, CDCl₃) δ 167.9, 146.9, 137.2, 131.4, 128.6, 128.0, 127.9, 127.5, 123.7, 121.9, 54.9, 43.75, 18.15.

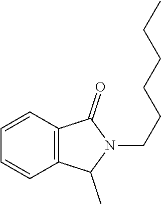 [SMILES: CCCCCCN1C(=O)c2ccccc2C1C]

### 2-hexyl-3-methylisoindolin-1-one (9hh)

The product was prepared using procedure F and isolated in 68% yield (0.471 g, 2.04 mmol) as a colorless oil.

¹H NMR (300 MHz, CDCl₃) δ 7.84-7.75 (m, 1H), 7.55-7.44 (m, 1H), 7.43-7.34 (m, 2H), 4.52 (q, J=6.7 Hz, 1H), 3.91 (ddd, J=13.9, 8.8, 7.3 Hz, 1H), 3.17 (ddd, J=13.8, 8.6, 5.2 Hz, 1H), 1.75-1.46 (m, 2H), 1.46-1.31 (m, 4H), 1.28 (d, J=5.9 Hz, 5H), 0.85 (td, J=6.6, 3.2 Hz, 3H).

¹³C NMR (75 MHz, CDCl₃) δ 167.7, 146.7, 132.0, 131.1, 127.9, 123.4, 121.7, 55.3, 39.8, 31.6, 28.5, 26.7, 22.6, 18.3, 14.1.

HR-MS (ESI) m/z: [M+Na]+ calcd for C₁₅H21NONa+254.1515, found 254.1519.

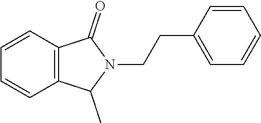 [SMILES: CC1c2ccccc2C(=O)N1CCc1ccccc1]

### 3-methyl-2-phenethylisoindolin-1-one (9hg)

The product was prepared using procedure F and isolated in 85% yield (0.640 g, 2.55 mmol) as a pale-yellow liquid.

¹H NMR (300 MHz, CDCl₃) δ 7.84 (d, J=7.4 Hz, 1H), 7.58-7.41 (m, 2H), 7.36 (d, J=7.4 Hz, 1H), 7.33-7.23 (m, 5H), 4.36 (q, J=6.7 Hz, 1H), 4.18 (ddd, J=14.6, 8.6, 6.5 Hz, 1H), 3.46 (ddd, J=14.4, 8.5, 6.4 Hz, 1H), 3.10-2.87 (m, 2H), 1.39 (d, J=6.7 Hz, 3H).

¹³C NMR (75 MHz, CDCl₃) δ 167.8, 146.8, 138.8, 131.9, 131.2, 128.7, 128.5, 127.9, 126.4, 123.4, 121.8, 55.9, 41.7, 34.9, 18.2.

HR-MS (ESI) m/z: [M+Na]+ calcd for C₁₇H17NONa+274.1202, found 274.1215.

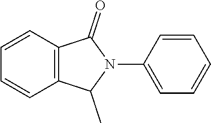 [SMILES: CC1c2ccccc2C(=O)N1c1ccccc1]

### 3-methyl-2-phenylisoindolin-1-one (9hm)

The product was prepared using procedure F and isolated in 70% yield (0.468 g, 2.1 mmol) as a yellow solid.

¹H NMR (300 MHz, CDCl₃) δ 7.93 (dt, J=7.6, 1.2 Hz, 1H), 7.66-7.38 (m, 7H), 7.29-7.17 (m, 1H), 5.20 (q, J=6.7 Hz, 1H), 1.45 (d, J=6.6 Hz, 3H).

¹³C NMR (75 MHz, CDCl₃) δ 166.8, 146.2, 137.0, 132.0, 131.7, 129.0, 128.3, 125.3, 124.1, 123.3, 121.9, 56.9, 18.9.

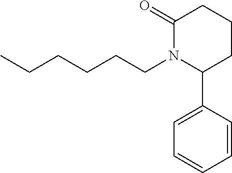 [SMILES: CCCCCCN1C(=O)CCCC1c1ccccc1]

### 1-hexyl-6-phenylpiperidin-2-one (9ih)

The product was prepared using procedure F and isolated in 74% yield (0.575 g, 2.22 mmol) as a pale-yellow liquid.

¹H NMR (300 MHz, CDCl₃) δ 7.48-6.82 (m, 5H), 4.62 (t, J=4.8 Hz, 1H), 3.98 (ddd, J=13.3, 9.1, 6.6 Hz, 1H), 2.61-2.26 (m, 3H), 2.12 (dddd, J=13.3, 10.9, 5.6, 3.9 Hz, 1H), 1.87 (ddq, J=13.6, 6.1, 3.4 Hz, 1H), 1.80-1.39 (m, 4H), 1.24 (qt, J=10.7, 6.5 Hz, 6H), 0.85 (q, J=7.2 Hz, 3H).

¹³C NMR (75 MHz, CDCl₃) δ 170.4, 141.5, 128.5, 127.4, 126.5, 60.8, 46.0, 32.3, 32.0, 31.7, 27.3, 26.7, 22.7, 17.0, 14.1.

HR-MS (ESI) m/z: [M+Na]+ calcd for C₁₇H25NONa+282.1828, found 282.1825.

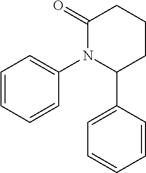 [SMILES: O=C1CCCC(c2ccccc2)N1c1ccccc1]

### 1,6-diphenylpiperidin-2-one (9im)

The product was prepared using procedure F and isolated in 71% yield (0.534 g, 2.13 mmol) as a yellow liquid.

¹H NMR (300 MHz, CDCl₃) δ 7.42-6.98 (m, 10H), 5.00 (t, J=5.1 Hz, 1H), 2.75-2.66 (m, 2H), 2.43-2.26 (m, 2H), 2.11-1.72 (m, 2H).

¹³C NMR (75 MHz, CDCl₃) δ 170.9, 142.1, 141.1, 128.7, 128.4, 127.4, 127.2, 126.9, 126.7, 65.2, 32.6, 32.3, 17.6.

HR-MS (ESI) m/z: [M+Na]+ calcd for C₁₇H17NONa+274.1202, found 274.1203.

We claim:

1. A process for direct reductive amination of a carbonyl compound, which process comprises reacting the carbonyl compound with an amine in the presence of a trifluoroacetoxyborane-amine (TFAB-amine) complex.

2. The process of claim 1, wherein the carbonyl compound is an aldehyde, a ketone, or a keto acid.

3. The process of claim 2, wherein the carbonyl compound is an aldehyde of formula (3): 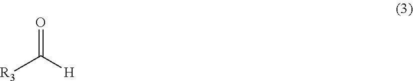
wherein, R is hydrogen, alkyl, trifluoromethyl, cycloalkyl, heteroalkyl, heterocycloalkyl, aryl, heteroaryl, heteroarylalkyl, arylalkenyl, or arylalkyl, wherein alkyl, cycloalkyl, aryl, and heteroaryl are optionally substituted.

4. The process of claim 2, wherein the carbonyl compound is a ketone of formula (5): 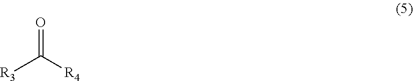
wherein, R₃ and R₄ are independently selected from the group consisting of hydrogen, alkyl, trifluoromethyl, cycloalkyl, heteroalkyl, heterocycloalkyl, aryl, heteroaryl, heteroarylalkyl, arylalkenyl, and arylalkyl, wherein alkyl, cycloalkyl, aryl, and heteroaryl are optionally substituted; or R₃ and R₄ are linked via an alkyl chain.

5. The process of claim 2, wherein the carbonyl compound is a ketoacid of formula (8): 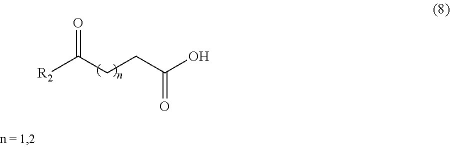
wherein, R₂ is hydrogen, alkyl, trifluoromethyl, cycloalkyl, heteroalkyl, heterocycloalkyl, aryl, heteroaryl, heteroarylalkyl, arylalkenyl, or arylalkyl, wherein alkyl, cycloalkyl, aryl, and heteroaryl are optionally substituted; and n is 1 or 2.

6. The process of claim 1, wherein the amine is an amine of formula (4): 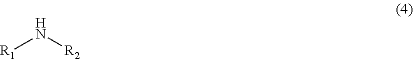
wherein R₁ and R are independently selected from the group consisting of hydrogen, alkyl, trifluoromethyl, cycloalkyl, heteroalkyl, heterocycloalkyl, aryl, heteroaryl, heteroarylalkyl, arylalkenyl, and arylalkyl, wherein alkyl, cycloalkyl, aryl, and heteroaryl are optionally substituted; or R₁ and R₂ are linked via an alkyl chain.

7. The process of claim 1, wherein the TFAB-amine complex is represented by formula (2): 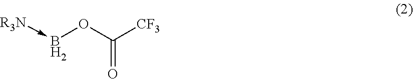
wherein, R₃N is NH₃, NH₂Me, NH₂CH₂Ph, NHMe₂, NH(i-Pr)₂, piperidine, NMe₃, NEt₃, pyridine, or 2-picoline.

8. The process of claim 7, wherein the TFAB-amine complex is TFAB-NH₃ or TFAB-NEt₃.

9. The process of claim 1, wherein the process of direct reductive amination is carried out in an organic solvent.

10. The process of claim 9, wherein the organic solvent is selected from tetrahydrofuran (THF), toluene, and dichloromethane (DCM).

11. The process of claim 1, wherein the process of direct reductive amination is carried out at temperature range from about room temperature to about reflux.

12. A trifluoroacetoxyborane-amine (TFAB-amine complex) of formula (2): 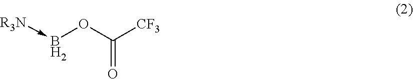
wherein R₃N is NH₃, NH₂Me, NH₂Bn, NHMe₂, NH(i-Pr)₂, piperidine, NMe₃, NEt₃, pyridine, or 2-picoline.

13. A process for preparation of a TFAB-amine complex of formula (2) of claim 12, which process comprises reacting a borane-amine with trifluoroacetic acid in the presence of a solvent.

14. The process of claim 13, wherein the borane-amine is represented by formula (1):
R₃N—BH₃ (1)
wherein, R₃N is NH₃, NH₂Me, NH₂CH₂Ph, NHMe₂, NH(i-Pr)₂, piperidine, NMe₃, NEt₃, pyridine, or 2-picoline.

15. The process of claim 13, wherein the process is carried out at a temperature from about RT to about reflux.

16. The process of claim 13, wherein the solvent is selected from THF, toluene, and DCM.

17. The process of claim 5, wherein the keto acids undergo tandem reductive amination-cycloamidation to obtain aromatic and aliphatic lactams.
